(19) United States
(12) Patent Application Publication
(10) Pub. No.: US 2003/0199558 A1
(43) Pub. Date: Oct. 23, 2003

(54) Hydroxamic acid and its derivatives as inhibitors of melanocyte tyrosinase for topical skin lighteners

(75) Inventors: Thomas P. Dooley, Vestavia Hills, AL (US); Lin Cheng, Huntsville, AL (US)

Correspondence Address: KING & SPALDING, 191 PEACHTREE STREET, N.E., ATLANTA, GA, 30303-1763, US

(73) Assignee: Integriderm, Inc.

(21) Appl. No.: 10/328,404

(22) Filed: Dec. 23, 2002

Related U.S. Application Data
(60) Provisional application No. 60/344,464, filed on Dec. 28, 2001.

(51) Int. Cl. A61K 31/455; A61K 31/19

(52) U.S. Cl. 514/354; 514/575

(57) ABSTRACT

Methods, compounds, and formulations are provided to reduce pigmentation in mammalian skin, comprising hydroxamic acid and its derivatives, and especially benzohydroxamic acid and its derivatives. The compounds preferably inhibit pigment synthesis in melanocytes through inhibition of melanocyte tyrosinase. The methods can be used for lightening skin, and for treating uneven skin complexions, which result from hyperpigmentation-related medical conditions such as melasma, age spots, freckles, ochronosis, and lentigo. The compounds can be used medically or cosmetically, and preferably as topical formulations.

### RELATIONSHIP TO PRIOR APPLICATIONS

This application claims priority to U.S. Provisional Patent Application No. 60/344,464, filed Dec. 28, 2001.

### FIELD OF THE INVENTION

The present invention relates to compounds and methods for inhibiting melanocyte tyrosinase and lightening the color of mammalian skin.

### BACKGROUND OF THE INVENTION

Melanogenesis is the process of production and subsequent distribution of melanin by melanocytes within the skin and hair follicles [1, 2]. Melanocytes have specialized lysosome-like organelles, termed melanosomes, which contain several enzymes that mediate the production of melanin. The copper-containing enzyme tyrosinase catalyzes the oxidation of the amino acid tyrosine into DOPA and subsequently DOPA-quinone. At least two additional melanosomal enzymes are involved in the eumelanogenesis pathway that produces brown and black pigments, including TRP-1 (DHICA oxidase), and TRP-2 (DOPAchrome tautomerase). Depending on the incorporation of a sulfur-containing reactant (e.g. cysteine or glutathione) into the products, the melanogenesis pathway diverges to produce pheomelanins (amber and red pigments).

The perceived color of skin and hair is determined by the ratio of eumelanins to pheomelanins, and in part on blood within the dermis. The balance in skin hue is genetically regulated by many factors, including but not limited to: (a) the levels of expression of tyrosinase, TRP-2, and TRP-1; (b) thiol conjugation (e.g. with glutathione or cysteine) leading to the formation of pheomelanins; (c) the α-melanocyte-stimulating hormone (α-MSH) and melanocortin receptor, which is coupled to the adenylate cyclase/protein kinase A pathway; [15] (d) the product of the agouti locus, agouti signal protein, which has been documented to down-regulate pigmentation of hair melanocytes in rodents; [16] and (e) yet unknown mechanisms that regulate the uptake and distribution of melanosomes in recipient epidermal and hair matrix keratinocytes. [2, 13, 14]

Abnormalities of human skin pigmentation occur as a result of both genetic and environmental factors. Exposure of skin (especially Caucasian) to ultraviolet radiation, particularly in the UVB (i.e. intermediate) wavelengths, upregulates synthesis of melanocyte tyrosinase resulting in increased melanogenesis and thus tanning. However, acute or persistent UVB exposure can result in the formation of hyperpigmented lesions or regions of skin, including malignant melanoma skin cancer. [17] Both actinic damage and constitutional abnormalities can produce affected regions such as melasma, age spots, liver spots, freckles and other lentigenes. [3, 18, 19]

Vitiligo is the converse of hyperpigmentation, in which cutaneous melanocytes are either ablated or fail to produce sufficient pigment. [17, 18, 20] Although it would be desirable to restore lost pigmentation in vitiligo-affected skin with topical therapies, this has proven to be quite difficult to accomplish in a high proportion of subjects. As an alternative to PUVA (psoralin-ultraviolet A) therapy or cosmetic camouflage with dihydroxyacetone sunless-tanning lotions, [18] one might reduce the normal pigmentation of the unaffected skin to reduce contrast. Furthermore, a global market demand has developed for skin-lightening agents as “vanity” cosmeccutical products, because lighter skin color is preferred by some dark-skinned individuals in many countries and races, for psychological or sociological reasons. [4, 5]

Some purportedly “active” or “functional” agents for lightening skin color (e.g. arbutin, kojic acid, niacinamide, licorice, magnesium ascorbyl phosphate, among others) have not been demonstrated yet to be clinically efficacious when critically analyzed in carefully controlled studies [5, 6, 25]. The U.S. FDA-approved pharmaceutical products containing 2-4% hydroquinone (“HQ”) are minimally to moderately efficacious. However, HQ has been demonstrated to be cytotoxic to cultured mammalian melanocytes, and mutagenic in Salmonella and mammalian Chinese hamster V79 cells [3-6, 10, 11, 25]. HQ appears to be an important intermediate in the bioactivation of the carcinogen benzene [12]. Although it has been repeatedly asserted in the dermatologic literature for many years, without substantiation, that HQ is an inhibitor of tyrosinase, this compound is not an effective inhibitor of the mammalian enzyme [5, 6, 25]. Hydroquinone's in vitro mechanism of action appears to be primarily a melanocytic cytotoxic effect. Its clinical mechanism of action on whole skin remains uncertain. Furthermore, as a result of concerns over safety, HQ is no longer considered as acceptable for use in Europe. In view of the disadvantages of the current industry standard skin-bleaching agent, HQ, it is highly desirable to identify other compounds with improved efficacy and safety characteristics, especially since a global demand is present in the marketplace.

Benzimidazolethiols have been studied and applied in many industrial fields. The most common application of benzimidazolethiols are as antioxidants in natural rubber, synthetic elastomers, and thermoplastics [34-35]. The affinity and hydrophobic chromatography of mushroom tyrosinase on benzimidazolethiols coupled on solid support have been studied, implying that benzimidazolethiols are a potential tyrosinase inhibitor [36]. Two filed (but abandoned) patent applications by a Japanese company disclose a number of benzimidazolethiols compounds, which allegedly are active as tyrosinase inhibitors [37]. Those compounds have not been either published or developed as commercially available topical skin depigmenting or lightening products to date.

Dooley et al., WO 01/64206 (published Sep. 7, 2001), discloses a series of compound classes including benzimidazolethiols, phenylthioureas, phenylthiols, bi- and multicyclic phenols, thiopheneamines, benzothiamides, and phenylamine, which are effective inhibitors of mammalian tyrosinase enzyme for use as skin lightening agents. The publication reports three in vitro properties, i.e., tyrosinase inhibition, pigment inhibition and toxicity in cultured melanocytes, for a number of benzimidazolthiols, thiophenols, phenylthioureas, of the following general structures:

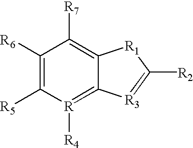

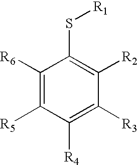

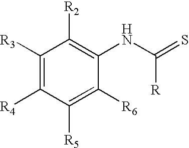

In addition, the publication reports tyrosinase inhibition, pigment inhibition and toxicity in cultured melanocytes, for a number of miscellaneous compounds described by the following structures:

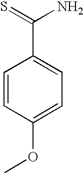

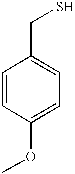

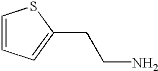

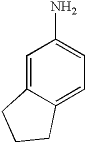

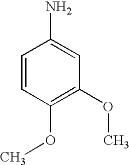

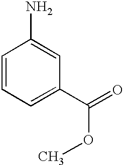

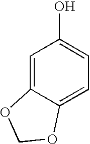

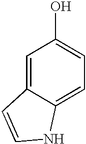

Benzohydroxamic acid is characterized generally by the following chemical structure:

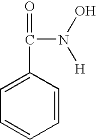

Benzohydroxamic acid and its derivatives have received varying commercial attention over the years. For example, in 2000 they were explored for use as a photographic material [38]. In other less recent years, this compound has been found to be an inhibitor of matrix metalloproteinase [39], ribonucleotide reductase [40], urease [41] and lipoxygenase [42].

Benzohydroxamic acid has not been investigated as a mammalian tyrosinase inhibitor, although two publications two decades ago mentioned that the compound and some specific derivatives thereof inhibited the activity of mushroom tyrosinase [43-44]. Specifically, the references disclose such activity for benzohydroxamic acid, salicylhydroxamic acid, and m-chlorobenzohydroxamic acid.

Various substituted benzyhydroxamic acids and their cytotoxic action, including 4-nitro substitutions, 4-chloro substitutions, 4-methyl substitutions, 4-methoxy substitutions, 3,4-methoxy substitutions, and 3,4,5-methoxy substitutions have also been disclosed. [45]

U.S. Pat. No. 5,514,676 discloses amino-benzoid acids, including a 3,4-amino substituted benzohydroxamic acid, and discusses their utility for inhibiting nonenzymatic cross-linking (protein aging).

WO 98/55449 discloses hydroxamic acids that purportedly have anti-cancer and anti-parasitic properties, including a benzohydroxamic acid derivative substituted at the 4-position by —CHCHC(O)NH(OH).

WO 97/16439 discloses hydroxylamine derivatives that purportedly are useful for enhancing molecular chaperon production, specifically including a 5-substituted trifluoromethyl derivative of benzohydroxamic acid.

It is an object of the present invention to provide novel benzohydroxamic acid derivatives.

It is another object of the present invention to provide novel pharmaceutical compositions of benzohydroxamic acid and its derivatives.

It is a further object of this invention to provide methods and compositions for reducing pigmentation in the skin of mammals, including humans.

Another object is to provide methods and compositions for reducing pigmentation of skin for cosmetic, beauty-enhancing, or aesthetic effects.

It is another object to provide methods and compositions for treating hyperpigmentation-related medical conditions such as melasma, age spots, freckles, ochronosis, postinflammatory hyperpigmentation, lentigo, and other pigmented skin blemishes.

Another object of the present invention is to provide methods and compositions for inhibiting mammalian melanocyte tyrosinase, the rate-limiting enzyme in the production of melanin from tyrosine and DOPA.

An additional object of the invention is to provide antioxidant compositions that protect skin from oxidative damage, and/or to prevent oxidative decomposition of product formulations.

Another object is to facilitate discovery of compounds that inhibit mammalian tyrosinase in cell-free extracts from mammalian melanocyte or melanoma cells, using either a colorometric DOPA oxidation or a radiolabeled tyrosine or DOPA substrate assay (IC₅₀≦300 μM).

Another object is to facilitate discovery of compounds that inhibit de novo pigment production (synthesis and/or accumulation) in cultured mammalian melanocyte or melanoma cells (IC₅₀≦300 μM).

Another object is to facilitate evaluation of compounds for toxicity in mammalian melanocyte, melanoma, or other cell cultures (IC₅₀≧300 μM).

Another object is to provide composition of matter and/or identity of compounds that are efficacious and/or exhibit reduced toxicity using one or more of the bioassays described in other objects, with biochemical characteristics equivalent to or superior to hydroquinone or methyl gentisate.

Still another object is to provide synthesis of derivatives of active and/or functional compounds of the invention, including by organic synthesis, combinatorial chemistry, medicinal chemistry, X-ray crystallography, rational drug design, and other methods.

Another object is to provide the use of formulations of the present invention for cosmetic, cosmeceutical, over-the-counter drug, and prescription drug products.

Another object is to provide formulations of the present invention for the purpose of reducing or preventing pigmentation in hair, albeit during the biosynthesis of hair, as a result of blocking pigment production within the melanocytes of hair follicles.

Another object is to provide the active and/or functional compounds of the present invention for use in inhibiting tyrosinase or tyrosinase-like enzymes from non-mammalian species, for instance for use in the food science industry for the inhibition of enzymatic browning.

### SUMMARY OF THE INVENTION

Hydroxamic acid and its derivatives, and especially benzohydroxamic acid and its derivatives that are preferably substituted at the meta- and/or para-positions are provided that reduce or prevent the production of pigment by mammalian melanocytes. The compounds preferably inhibit the enzymatic activity of melanocyte tyrosinase, though some compounds may also control pigment production in melanocyte cells without necessarily being potent inhibitors of the enzyme. Therefore, the compounds can be used in applications wherein controlling or preventing the production of pigments in mammalian skin is desired. A few examples of such applications include:

1. As a vanity product, to lighten the skin of an individual, especially of dark skinned individuals;

2. To lessen the hue of pigmented skin blemishes such as freckles and age spots;

3. To diminish uneven pigmentation marks and surface color irregularities;

4. To treat hyperpigmentation-related medical conditions such as melasma, ochronosis, and lentigo;

5. To lighten hair pigmentation when applied to skin containing pigmented hair follicles;

6. To lessen postinflammatory hyperpigmentation resulting from trauma, acne, invasive surgery, a face lift, laser treatment, or cosmetic surgery; and

7. To reduce skin pigmentation in normal skin adjacent to areas affected by vitiligo, thereby diminishing the contrast in color between normal and vitiligo-affected skin.

Numerous hydroxamic acid and benzohydroxamic acid derivatives have been discovered with which the present invention can be practiced. These compounds exhibit activity in the mammalian tyrosinase and/or melanocyte cell culture pigmentation assays, yet with minimal (or no) cytotoxicity. These compounds exhibit characteristics that are equivalent to or superior to the known standard skin-bleaching agent, hydroquinone, or the known standard tyrosinase inhibitor, methyl gentisate.

The compounds are typically applied topically to the skin wherein tyrosinase activity is sought to be reduced through a lotion or occlusive patch. The compounds can be spread over a larger area to produce an even skin tone fade, or they can be applied locally to skin blemishes and other localized conditions to minimize skin irregularities. Moreover, because most of the compounds are selective against melanocyte tyrosinase, the compounds can also be administered systemically by methods including oral, intradermal, transdermal, intraveneous, and parenteral administrations. The product works by inhibiting the production of melanin in cells beneath the skin surface. Because the skin naturally renews itself about every 28 days, when the compounds of the present invention are administered old (differentiated) pigmented keratinocytes cells are gradually sloughed off and keratinocytes with less melanin are eventually brought to the surface giving the skin a lighter, more even toned complexion.

The hydroxamic acids employed in the practice of the present invention are preferably represented by the following structure (I):

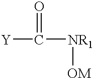

wherein:

M is a pharmaceutically acceptable cation, preferably hydrogen;

R₁ is hydrogen, or C₁-C₆ alkyl or cycloalkyl, preferably hydrogen or lower alkyl, and most preferably hydrogen; and

Y is substituted or unsubstituted cycloalkyl, aryl, heterocycle, or heteroaryl, which is preferably mono- or di-substituted at the 3 and/or 4 carbon. Most preferably, Y is aryl or heteroaryl which is mono- or di-substituted at the 3 and/or 4 carbon positions by lower alkyl, hydroxy, NR₉R₉, lower alkoxy, phenoxy, halo, NHC(O)CH₃, and/or acetyl.

### DETAILED DESCRIPTION OF THE INVENTION

Discussion

As noted above, hydroxamic acid and benzohydroxamic acid derivatives for inhibiting or preventing melanin formation in skin have been discovered for the treatment of various melanin-associated conditions. For example, the compound can be used as a “vanity” product, to lighten the skin of an individual, especially of dark skinned individuals. Alternatively, the compound can be used to reduce uneven pigmentation marks and surface color irregularities, or to diminish pigmented skin blemishes such as freckles and age spots and hyperpigmentation-related medical conditions such as melasma, ochronosis, and lentigo. The compounds can also be used to lighten hair when applied to skin containing pigmented hair follicles, and to lessen postinflammatory hyperpigmentation resulting from trauma, acne, invasive surgery, a face lift, laser treatment, or cosmetic surgery. The active or functional compounds can also be used to reduce skin pigmentation in normal skin adjacent to areas affected by vitiligo, thereby diminishing the contrast in color between normal and vitiligo affected skin.

The invention thus provides a method for lightening mammalian skin that includes applying or otherwise administering an effective treatment amount of benzohydroxamic acid or a derivative thereof, or a pharmaceutically acceptable salt thereof, optionally in a pharmaceutically acceptable carrier, to a mammalian subject in need thereof. The invention also includes a pharmaceutical composition for topical or general systemic administration, including oral, intradermal, transdermal, occlusive patch, intraveneous, and parenteral formulations, that includes an effective amount of the pigmentation-inhibiting compound. The present invention is principally concerned with compositions that inhibit mammalian tyrosinase activity, and which thus have medicinal and/or cosmetic value. However, the present invention can also extend to compounds that inhibit melanin formation within melanocytes through mechanisms other than tyrosinase activity.

Many of the compounds may possess other activities that are beneficial when integrated into the compositions of the present invention. For example, many of the compounds may possess antioxidant properties, and thus can inhibit oxidative damage to the skin, or contribute to the stability of the formulation.

Compounds of the Present Invention

In a first principal embodiment the compounds of the present invention are hydroxamic acids and hydroxamic acid derivatives defined by the following structure (I)

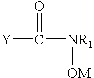

wherein:

M is a pharmaceutically acceptable cation, preferably hydrogen;

R₁ is hydrogen, or C₁-C₆ alkyl or cycloalkyl, preferably hydrogen or lower alkyl, and most preferably hydrogen; and

Y is substituted or unsubstituted cycloalkyl, aryl, heterocycle, or heteroaryl, which is preferably mono- or di-substituted at the 3 and/or 4 carbon. Most preferably, Y is aryl or heteroaryl which is mono- or di-substituted at the 3 and/or 4 carbon positions by lower alkyl, hydroxy, NR₉R₉, lower alkoxy, phenoxy, halo, NHC(O)CH₃, and/or acetyl.

In a second principal embodiment the compounds of the present invention are represented by the following structure (II):

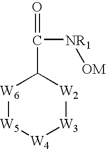

wherein:

M is a pharmaceutically acceptable cation;

R₁ is hydrogen, or C₁-C₆ alkyl or cycloalkyl;

W₂ is CR₂R_{2′}, NR², O or S; W₃ is CR₃R_{3′}, NR³, O or S; W₄ is CR₄R_{4′}, NR⁴, O or S; W₅ is CR₅R_{5′}, NR⁵, O or S; and W₆ is CR₆R_{6′}, NR⁶, O or S;

R₂, R₃, R₄, R₅, and R₆ are independently selected from (i) hydrogen, (ii) halogen, (iii) NO₂, (iv) —CN, (v) —OR₁₀ or phenoxy, (vi) —NHSO₂—C₁₋₃alkyl, (vii) —NHCO—C₁₋₅ alkyl, (viii) oxime, (ix) hydrazine, (x) —NR₉R₁₀, (xi) SO₂, (xii) SO₃, (xiii) —SR₁₀, (xiv) C₁₋₅ acyloxy, (xv) PO₃, (xvi) PO₄, (xvii) thiol, (xviii) —COOR₉, (xix) C₂₋₅ alkynyl, (xx) C(O)C₁₋₃ alkyl, and (xxi) —C₁₋₈ alkyl, —C₂₋₈ alkenyl, aryl, heteroaryl, or heterocycle, optionally substituted with one or more of —OH, —SH, C(O)H, COOR₉, C₁₋₅ acyloxy, halogen, NR₉R₁₀, C₁₋₅ thioether, or C₁₋₅ alkoxy;

R_{2′}, R_{3′}, R_{4′}, R_{5′}, and R_{6′} are independently H or a valence for bonding;

R², R³, R⁴, R⁵, and R⁶ are independently selected from (i) substituted or unsubstituted alkyl, alkenyl, aryl, or heterocycle, (ii) —C₁₋₅ alkoxy, (iii) —OH, (iv) hydrogen, (v) C(O)—C₁₋₃ alkyl, (vi) —(CH₂)₁₋₅C(O)NR₉R₁₀, or (vii) a valence for bonding;

alternatively, R₃ and R₄, or R₄ and R₅, combine to form a fused ring-structure which is cycloalkyl, aryl, heterocyclyl or heteroaryl selected from phenyl, cyclopentyl, cyclohexyl, pyrrole, furan, thiophene, pyrazole, pyridine, —X—(CH₂)—X—, or —(CH₂)₂X— wherein X is independently NH, S, or O;

R₉ is hydrogen or C₁₋₃ alkyl;

R₁₀ is hydrogen, C₁₋₈ alkyl, —C₂₋₈ alkenyl, —(CH₂)ₙOₘ(CH₂)_{n′}-aryl, —(CH₂)ₙOₘ(CH₂)_{n′}-heteroaryl, or —(CH₂)ₙOₘ(CH₂)_{n′}-heterocycle, optionally substituted with one or more of —OH, —SH, C(O)H, COOR₉, C₁₋₈ acyloxy, halogen, NR₉R₉, C₁₋₅ thioether, or C₁₋₅ alkoxy;

m is 0 or 1; and

n and n′ are independently 0, 1, 2, or 3.

In this second principal embodiment, M is preferably hydrogen, and R₁ is preferably lower alkyl and even more preferably hydrogen.

A first subembodiment of the second principal embodiment is defined when:

W₂ is CR₂R_{2′}, or NR²; W₃ is CR₃R_{3′}, or NR³; W₄ is CR₄R_{4′} or NR⁴; W₅ is CR₅R_{5′} or NR⁵; and W₆ is CR₆R_{6′}, or NR⁶;

R_{2′}, R_{3′}, R_{4′}, R_{5′}, and R_{6′} are a valence for bonding; and

R², R³, R⁴, R⁵, and R⁶ are a valence for bonding.

In this first subembodiment of the second principal embodiment, M is preferably hydrogen, and R₁ is preferably lower alkyl and even more preferably hydrogen.

A second subembodiment of the second principal embodiment is defined when:

W₂ is CR₂R_{2′}; W₃ is CR₃R_{3′}; W₄ is NR⁴; W₅ is CR₅R_{5′}; and W₆ is CR₆R_{6′};

R_{2′}, R_{3′}, R_{4′}, R_{5′}, and R_{6′} are a valence for bonding; and

R⁴ is a valence for bonding.

In this second subembodiment of the second principal embodiment, M is preferably hydrogen, and R₁ is preferably lower alkyl and even more preferably hydrogen.

A third subembodiment of the second principal embodiment is defined when:

W₂ is CR₂R_{2′}; W₃ is CR₃R_{3′}; W₄ is CR₄R_{4′}; W₅ is CR₅R_{5′}; and W₆ is CR₆R_{6′};

R_{2′}, R_{3′}, R_{4′}, R_{5′}, and R_{6′} are a valence for bonding; and

R₂, R₃, R₄, R₅ and R₆ are independently selected from (i) hydrogen, (ii) halogen, (iii) NO₂, (iv) —CN, (v) —OR₁₀ or phenoxy, (vi) —NR₉R₁₀, (vii) C₁₋₅ acyloxy, (viii) thiol, (ix) COOR₉, (x) C(O)C₁₋₃alkyl, (xi) —NHCO—C₁₋₅ alkyl, and (xii) —C₁₋₅ alkyl, —C₂₋₅ alkenyl, aryl, heteroaryl, or heterocycle, optionally substituted with one or more of —OH, —SH, C(O)H, COOR₉, C₁₋₅ acyloxy, halogen, NR₉R₁₀, C₁₋₅ thioether, or C₁₋₅ alkoxy.

In this third subembodiment of the second principal embodiment, M is preferably hydrogen, and R₁ is preferably lower alkyl and even more preferably hydrogen.

A fourth subembodiment of the second principal embodiment is defined when:

W₂ is CR₂R_{2′}; W₃ is CR₃R_{3′}; W₄ is CR₄R_{4′}; W₅ is CR₅R_{5′}; and W₆ is CR₆R_{6′};

R_{2′}, R_{3′}, R_{4′}, R_{5′}, and R_{6′} are a valence for bonding; and

R₂, R₃, R₄, R₅ and R₆ are independently selected from (i) hydrogen, (ii) halogen, (iii) NO₂, (iv) —CN, (v) —OR₉ or phenoxy, (v) —NR₉R₉, (vi) C₁₋₃ acyloxy, (vii) thiol, (viii) COOR₉, (x) C(O)C₁₋₃alkyl, (xi) —NHCO—C₁₋₃ alkyl, (xii) —C₁₋₃ alkyl, —C₂₋₃ alkenyl, aryl, heteroaryl, or heterocycle, optionally substituted with one or more of —OH, —SH, C(O)H, COOR₉, C₁₋₅ acyloxy, halogen, NR₉R₉, C₁₋₃ thioether, or C₁₋₃ alkoxy.

In this fourth subembodiment of the second principal embodiment, M is preferably hydrogen, and R₁ is preferably lower alkyl and even more preferably hydrogen.

A fifth subembodiment of the second principal embodiment is defined when:

W₂ is CR₂R_{2′}; W₃ is CR₃R_{3′}; W₄ is CR₄R_{4′}; W₅ is CR₅R_{5′}; and W₆ is CR₆R_{6′};

R_{2′}, R_{3′}, R_{4′}, R_{5′}, and R_{6′} are a valence for bonding; and

R₂, R₃, R₄, R₅ and R₆ are independently selected from (i) hydrogen, (ii) halogen, (iii) —OR₁₀ or phenoxy, (iv) —NR₉R₉, (v) thiol, (vi) C(O)C₁₋₃alkyl, (vii) —NHCO—C₁₋₃ alkyl, and (viii) —C₁₋₃ alkyl or C₂₋₃ alkenyl optionally substituted with one or more of —OH, —SH, halogen, and NH₂.

In this fifth subembodiment of the second principal embodiment, M is preferably hydrogen, and R₁ is preferably lower alkyl and even more preferably hydrogen.

A sixth subembodiment of the second principal embodiment is defined when:

W₂ is CR₂R_{2′}; W₃ is CR₃R_{3′}; W₄ is CR₄R_{4′}; W₅ is CR₅R_{5′}; and W₆ is CR₆R_{6′};

R_{2′}, R_{3′}, R_{4′}, R_{5′}, and R_{6′} are a valence for bonding; and

R₂, R₃, R₄, R₅ and R₆ are independently selected from hydrogen, lower alkyl, hydroxy, NR₉R₉, lower alkoxy, phenoxy, halo, NHC(O)CH₃, and acetyl.

In this sixth subembodiment of the second principal embodiment, M is preferably hydrogen, and R₁ is preferably lower alkyl and even more preferably hydrogen.

A seventh subembodiment of the second principal embodiment is defined when:

W₂ is CR₂R_{2′}; W₃ is CR₃R_{3′}; W₄ is CR₄R_{4′}; W₅ is CR₅R_{5′}; and W₆ is CR₆R_{6′};

R_{2′}, R_{3′}, R_{4′}, R_{5′}, and R_{6′} are a valence for bonding; and

R₃ and R₄, or R₄ and R₅, combine to form a fused ring-structure which is cycloalkyl, aryl, heterocyclyl, or heteroaryl selected from phenyl, cyclopentyl, cyclohexyl, pyrrole, furan, thiophene, pyrazole, pyridine, —X—(CH₂)—X—, or —(CH₂)₂X— wherein X is independently NH, S, or O.

In this seventh subembodiment of the second principal embodiment, M is preferably hydrogen, and R₁ is preferably lower alkyl and even more preferably hydrogen.

In a third principal embodiment the compounds of the present invention are defined by the following structure (III):

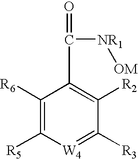

wherein:

M is a pharmaceutically acceptable cation;

R₁ is hydrogen, or C₁-C₆ alkyl or cycloalkyl;

W₄ is CR₄ or N;

R₂, R₃, R₄, R₅, and R₆ are independently selected from (i) hydrogen, (ii) halogen, (iii) NO₂, (iv) —CN, (v) —OR₁₀ or phenoxy, (vi) —NHSO₂—C₁₋₃alkyl, (vii) —NHCO—C₁₋₅ alkyl, (viii) oxime, (ix) hydrazine, (x) —NR₉R₁₀, (xi) SO₂, (xii) SO₃, (xiii) SR₁₀, (xiv) C₁₋₅ acyloxy, (xv) PO₃, (xvi) PO₄, (xvii) thiol, (xviii) —COOR₉, (xix) C₂₋₅ alkynyl, (xx) C(O)C₁₋₃ alkyl, and (xxi) —C₁₋₈ alkyl, —C₂₋₈ alkenyl, aryl, heteroaryl, or heterocycle, optionally substituted with one or more of —OH, —SH, C(O)H, COOR₉, C₁₋₅ acyloxy, halogen, NR₉R₁₀, C₁₋₅ thioether, or C₁₋₅ alkoxy;

alternatively, R₃ and R₄, or R₄ and R₅, combine to form a fused ring-structure which is cycloalkyl, aryl, heterocyclyl or heteroaryl selected from phenyl, cyclopentyl, cyclohexyl, pyrrole, furan, thiophene, pyrazole, pyridine, —X—(CH₂)—X—, or —(CH₂)₂X— wherein X is independently NH, S, or O;

R₉ is hydrogen or C₁₋₃ alkyl;

R₁₀ is hydrogen, C₁₋₈ alkyl, —C₂₋₈ alkenyl, —(CH₂)ₙOₘ(CH₂)_{n′}-aryl, —(CH₂)ₙOₘ(CH₂)_{n′}-heteroaryl, or —(CH₂)ₙOₘ(CH₂)_{n′}-heterocycle, optionally substituted with one or more of —OH, —SH, C(O)H, COOR₉, C₁₋₈ acyloxy, halogen, NR₉R₉, C₁₋₅ thioether, or C₁₋₅ alkoxy;

m is 0 or 1; and

n and n′ are independently 0, 1, 2, or 3.

In this third principal embodiment, M is preferably hydrogen, and R₁ is preferably lower alkyl and even more preferably hydrogen.

A first subembodiment of the third principal embodiment is defined when:

W₄ is N.

In this first subembodiment of the third principal embodiment, M is preferably hydrogen, and R₁ is preferably lower alkyl and even more preferably hydrogen.

A second subembodiment of the third principal embodiment is defined when:

W₄ is CR₄; and

R₂, R₃, R₄, R₅ and R₆ are independently selected from (i) hydrogen, (ii) halogen, (iii) NO₂, (iv) —CN, (v) —OR₁₀ or phenoxy, (vi) —NR₉R₁₀, (vii) C₁₋₅ acyloxy, (viii) thiol, (ix) COOR₉, (x) C(O)C₁₋₃alkyl, (xi) —NHCO—C₁₋₅ alkyl, and (xii) —C₁₋₅ alkyl, —C₂₋₅ alkenyl, aryl, heteroaryl, or heterocycle, optionally substituted with one or more of —OH, —SH, C(O)H, COOR₉, C₁₋₅ acyloxy, halogen, NR₉R₁₀, C₁₋₅ thioether, or C₁₋₅ alkoxy.

In this second subembodiment of the third principal embodiment, M is preferably hydrogen, and R₁ is preferably lower alkyl and even more preferably hydrogen.

A third subembodiment of the third principal embodiment is defined when:

W₄ is CR₄;

R₂, R₃, R₄, R₅ and R₆ are independently selected from (i) hydrogen, (ii) halogen, (iii) NO₂, (iv) —CN, (v) —OR₉ or phenoxy, (v) —NR₉R₉, (vi) C₁₋₃ acyloxy, (vii) thiol, (viii) COOR₉, (x) C(O)C₁₋₃alkyl, (xi) —NHCO—C₁₋₃ alkyl, (xii) —C₁₋₃ alkyl, —C₂₋₃ alkenyl, aryl, heteroaryl, or heterocycle, optionally substituted with one or more of —OH, —SH, C(O)H, COOR₉, C₁₋₅ acyloxy, halogen, NR₉R₉, C₁₋₃ thioether, or C₁₋₃ alkoxy.

In this third subembodiment of the third principal embodiment, M is preferably hydrogen, and R₁ is preferably lower alkyl and even more preferably hydrogen.

A fourth subembodiment of the third principal embodiment is defined when:

W₄ is CR₄; and

R₂, R₃, R₄, R₅ and R₆ are independently selected from (i) hydrogen, (ii) halogen, (iii) —OR₁₀ or phenoxy, (iv) —NR₉R₉, (v) thiol, (vi) C(O)C₁₋₃alkyl, (vii) —NHCO—C₁₋₃ alkyl, and (viii) —C₁₋₃ alkyl or —C₂₋₃ alkenyl optionally substituted with one or more of —OH, —SH, halogen, and NH₂.

In this fourth subembodiment of the third principal embodiment, M is preferably hydrogen, and R₁ is preferably lower alkyl and even more preferably hydrogen.

A fifth subembodiment of the third principal embodiment is defined when:

W₄ is CR₄; and

R₂, R₃, R₄, R₅ and R₆ are independently selected from lower alkyl, hydroxy, NR₉R₉, lower alkoxy, phenoxy, halo, NHC(O)CH₃, and acetyl.

In this fifth subembodiment of the third principal embodiment, M is preferably hydrogen, and R₁ is preferably lower alkyl and even more preferably hydrogen.

A sixth subembodiment of the third principal embodiment is defined when:

W₄ is CR₄;

R₃ and R₄, or R₄ and R₅, combine to form a fused ring-structure which is cycloalkyl, aryl, heterocyclyl, or heteroaryl selected from phenyl, cyclopentyl, cyclohexyl, pyrrole, furan, thiophene, pyrazole, pyridine, —X—(CH₂)—X—, or —(CH₂)₂X— wherein X is independently NH, S, or O.

In this sixth subembodiment of the third principal embodiment, M is preferably hydrogen, and R₁ is preferably lower alkyl and even more preferably hydrogen.

In a fourth principal embodiment the compounds of the present invention are defined by one of structures (IV)-(XXIX):

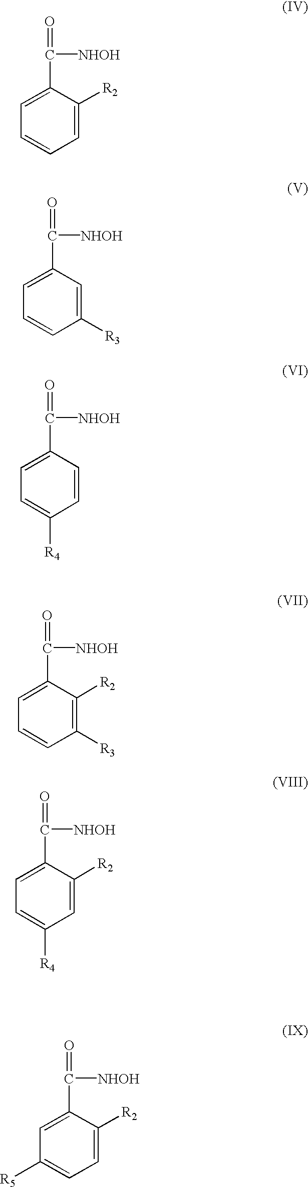

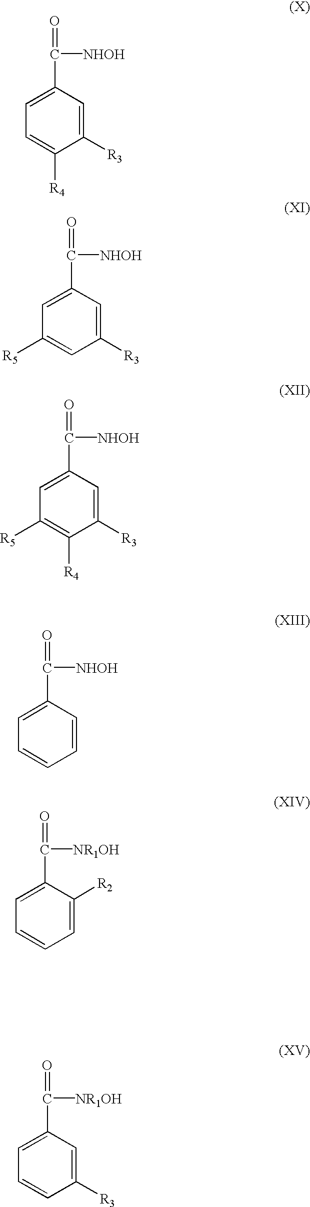

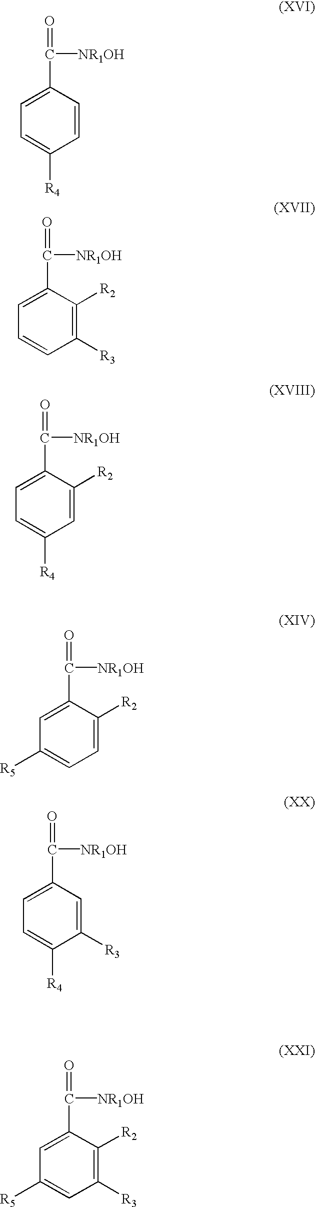

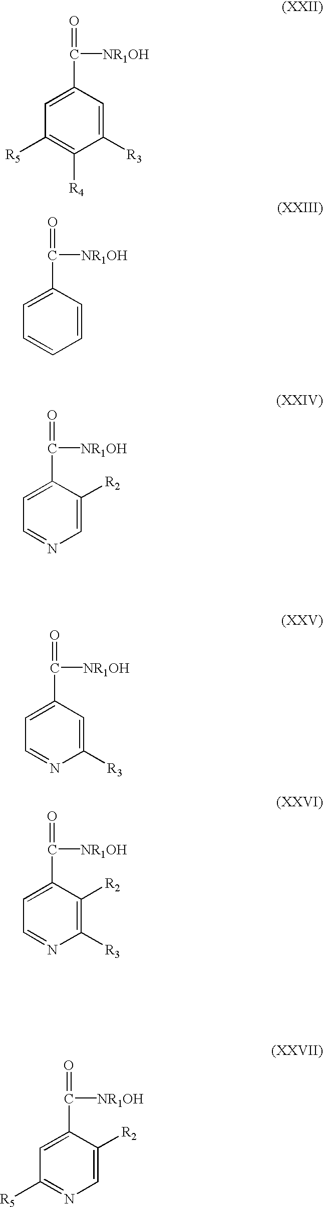

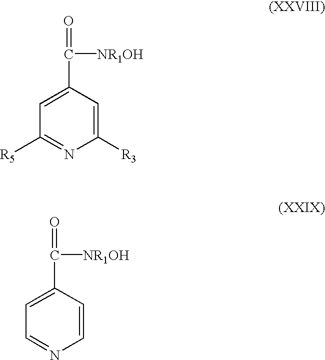

or a pharmaceutically acceptable salt thereof, wherein:

R₁ is hydrogen, or C₁-C₆ alkyl or cycloalkyl;

R₂, R₃, R₄, R₅, and R₆ are independently selected from (i) hydrogen, (ii) halogen, (iii) NO₂, (iv) —CN, (v) —OR₁₀ or phenoxy, (vi) —NHSO₂—C₁₋₃alkyl, (vii) —NHCO—C₁₋₅ alkyl, (viii) oxime, (ix) hydrazine, (x) —NR₉R₁₀, (xi) SO₂, (xii) SO₃, (xiii) SR₁₀, (xiv) C₁₋₅ acyloxy, (xv) PO₃, (xvi) PO₄, (xvii) thiol, (xviii) —COOR₉, (xix) C₂₋₅ alkynyl, (xx) C(O)C₁₋₃ alkyl, and (xxi) —C₁₋₈ alkyl, —C₂₋₈ alkenyl, aryl, heteroaryl, or heterocycle, optionally substituted with one or more of —OH, —SH, C(O)H, COOR₉, C₁₋₅ acyloxy, halogen, NR₉R₁₀, C₁₋₅ thioether, or C₁₋₅ alkoxy;

alternatively, R₃ and R₄, or R₄ and R₅, combine to form a fused ring-structure which is cycloalkyl, aryl, heterocyclyl or heteroaryl selected from phenyl, cyclopentyl, cyclohexyl, pyrrole, furan, thiophene, pyrazole, pyridine, —X—(CH₂)—X—, or —(CH₂)₂X— wherein X is independently NH, S, or O;

R₉ is hydrogen or C₁₋₃ alkyl;

R₁₀ is hydrogen, C₁₋₈ alkyl, —C₂₋₈ alkenyl, —(CH₂)ₙOₘ(CH₂)_{n′}-aryl, —(CH₂)ₙOₘ(CH₂)_{n′}-heteroaryl, or —(CH₂)_{n′}Oₘ(CH₂)_{n′}-heterocycle, optionally substituted with one or more of —OH, —SH, C(O)H, COOR₉, C₁₋₈ acyloxy, halogen, NR₉R₉, C₁₋₅ thioether, or C₁₋₅ alkoxy;

m is 0 or 1; and

n and n′ are independently 0, 1, 2, or 3.

In a first subembodiment of this fourth principal embodiment,

R₁ is hydrogen, or C₁-C₆ alkyl or cycloalkyl; and

R₂, R₃, R₄, R₅ and R₆ are independently selected from (i) hydrogen, (ii) halogen, (iii) NO₂, (iv) —CN, (v) —OR₁₀ or phenoxy, (vi) —NR₉R₁₀, (vii) C₁₋₅ acyloxy, (viii) thiol, (ix) COOR₉, (x) C(O)C₁₋₃alkyl, (xi) —NHCO—C₁₋₅ alkyl, and (xii) —C₁₋₅ alkyl, —C₂₋₅ alkenyl, aryl, heteroaryl, or heterocycle, optionally substituted with one or more of —OH, —SH, C(O)H, COOR₉, C₁₋₅ acyloxy, halogen, NR₉R₁₀, C₁₋₅ thioether, or C₁₋₅ alkoxy.

In a second subembodiment of the fourth principal embodiment,

R₁ is hydrogen or lower alkyl; and

R₂, R₃, R₄, R₅ and R₆ are independently selected from (i) hydrogen, (ii) halogen, (iii) NO₂, (iv) —CN, (v) —OR₉ or phenoxy, (v) —NR₉R₉, (vi) C₁₋₃ acyloxy, (vii) thiol, (viii) COOR₉, (x) C(O)C₁₋₃alkyl, (xi) —NHCO—C₁₋₃ alkyl, (xii) —C₁₋₃ alkyl, —C₂₋₃ alkenyl, aryl, heteroaryl, or heterocycle, optionally substituted with one or more of —OH, —SH, C(O)H, COOR₉, C₁₋₅ acyloxy, halogen, NR₉R₉, C₁₋₃ thioether, or C₁₋₃ alkoxy.

In a third subembodiment of the fourth principal embodiment,

R₁ is hydrogen or lower alkyl; and

R₂, R₃, R₄, R₅ and R₆ are independently selected from (i) hydrogen, (ii) halogen, (iii) —OR₁₀ or phenoxy, (iv) —NR₉R₉, (v) thiol, (vi) C(O)C₁₋₃alkyl, (vii) —NHCO—C₁₋₃ alkyl, and (viii) —C₁₋₃ alkyl or C₂₋₃ alkenyl optionally substituted with one or more of —OH, —SH, halogen, and NH₂.

In a fourth subembodiment of the fourth principal embodiment,

R₁ is hydrogen or lower alkyl; and

R₂, R₃, R₄, R₅ and R₆ are independently selected from lower alkyl, hydroxy, NR₉R₉, lower alkoxy, phenoxy, halo, NHC(O)CH₃, and acetyl.

In a fifth subembodiment of the fourth principal embodiment,

R₁ is hydrogen or lower alkyl; and

R₂, R₃, R₄, R₅ and R₆ are independently selected from methyl, ethyl, methoxy, butoxy, phenoxy, hydroxy, NH₂, N(Me)₂, and halo.

In a sixth subembodiment of the fourth principal embodiment,

R₁ is hydrogen; and

R₂, R₃, R₄, R₅ and R₆ are methyl.

In a seventh subembodiment of the fourth principal embodiment,

R₁ is hydrogen; and

R₂, R₃, R₄, R₅ and R₆ are methoxy.

In an eighth subembodiment of the fourth principal embodiment,

R₁ is hydrogen; and

R₂, R₃, R₄, R₅ and R₆ are hydroxy.

In a ninth subembodiment of the fourth principal embodiment,

R₁ is hydrogen; and

R₂, R₃, R₄, R₅ and R₆ are NH₂.

In a tenth subembodiment of the fourth principal embodiment,

R₁ is hydrogen; and

R₂, R₃, R₄, R₅ and R₆ are N(Me)₂.

In an eleventh subembodiment of the fourth principal embodiment,

R₁ is hydrogen; and

R₂, R₃, R₄, R₅ and R₆ are halo.

In a twelth subembodiment of the fourth principal embodiment,

R₁ is hydrogen; and

R₂, R₃, R₄, R₅ and R₆ are butoxy.

In a thirteenth subembodiment of the fourth principal embodiment,

R₁ is hydrogen; and

R₂, R₃, R₄, R₅ and R₆ are phenoxy.

In a fifth principal embodiment the compounds of the present invention are selected from one of the compounds recited in the following Table I:

TABLE I

| ID # | Structure | Compound's Name |
|---|---|---|
| ID-357 | 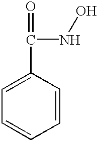 | Benzohydroxamic acid |
| ID-483 | 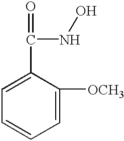 | 2-Methoxybenzohydroxamic acid |
| ID-480 | 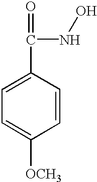 | 4-Methoxybenzohydroxamic acid |
| ID-479 | 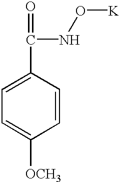 | Potassium salt of 4- methoxybenzohydroxamic acid |
| ID-497 | 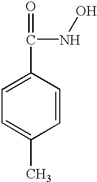 | 4-Methylbenzohydroxamic acid |
| ID-478 | 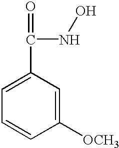 | 3-Methoxybenzohydroxamic acid |
| ID-500 | 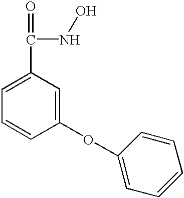 | 3-Phenoxybenzohydroxamic acid |
| ID-482 | 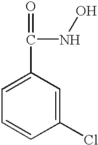 | 3-Chlorobenzohydroxamic acid |
| ID-481 | 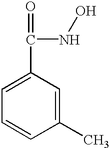 | 3-Methylbenzohydroxamic acid |
| ID-485 | 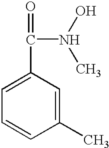 | 3,N-Dimethylbenzohydroxamic acid |
| ID-461 | 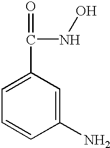 | 3-Aminobenzohydroxamic acid |
| ID-486 | 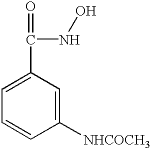 | 3-Acetamidobenzohydroxamic acid |
| ID-499 | 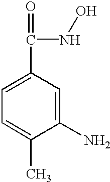 | 13-Amino-4-methyl- benzohydroxamic acid |
| ID-476 | 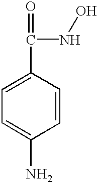 | 4-Aminobenzohydroxamic acid |
| ID-498 | 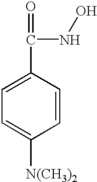 | 4-Dimethylaminobenzo- hydroxamic acid |
| ID-318 | 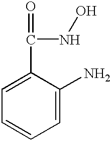 | 2-Aminobenzohydroxamic acid |
| ID-484 | 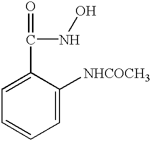 | 2-Acetamidobenzohydroxamic acid |
| ID-282 | 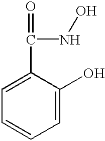 | Salicylhydroxamic acid |
| ID-477 | 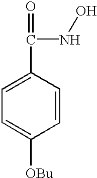 | 4-Butoxybenzohydroxamic acid |
| ID-456 | 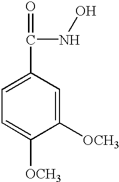 | 3,4-Dimethoxybenzohydroxamic acid |
| ID-458 | 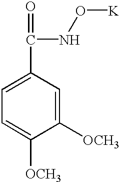 | Potassium salt of 3,4-dimethoxy- benzohydroxamic acid |

The compounds of this invention can be optionally substituted, and in several instances in this document the compounds are specifically decribed as substituted or unsubstituted. Although it will be understood that the substituents include all substituents that do not adversely affect the activity of the compound as a skin lightener, in one series of embodiments, the substituents are selected from alkyl (including lower alkyl), heteroalkyl, aryl, heterocyclic (including heteroaryl and heterocycloalkyl), halo, hydroxyl, carboxyl, acyl, acyloxy, amino, alkylamino, arylamino, alkoxy, aryloxy, alkylthio, alkylamido, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991. In another series of embodiments the substituents are selected from —OH, —SH, C(O)H, COOR9, C1-5 acyloxy, halogen, NR9R10, C1-5 thioether, or C1-5 alkoxy.

It will be understood that the present invention also covers “prodrugs” for such compositions, and pharmaceutically acceptable salts thereof.

Some generalizations can be made about the foregoing compounds, and preferred structures therefore. For example, each of the various embodiments and subembodiments can be further limited as follows:

the benzohydroxamic acid is substituted only at the meta position;

the benzohydroxamic acid is substituted only at the para position;

the benzohydroxamic acid is substituted at the meta and para positions;

the benzohydroxamic acid is substituted only at the meta and/or para position, and the substituting moiety comprises less than 17, 11, 9, 7, 5, 4, 3, or 2 carbon or heteroatoms;

the benzohydroxamic acid is substituted only at the meta and/or para position, the substituting moiety comprises less than 7 atoms or heteroatoms, and the substituting mocity is not one or any of OH, NH₂, dimethylamino, phenyl, nitro, halo, methyl, butyl, methoxy, butoxy, propoxy, alkene, trihalomethyl, Sme, C(O)Ome, C(O)C(CH₃)₃, and/or CH₂Cl;

the benzohydroxamic acid is substituted at the meta position by hydroxy, methoxy, amino, dimethylamino, halo, methyl, phenoxy, or butoxy;

the benzohydroxamic acid is substituted at the para position by hydroxy, methoxy, amino, dimethylamino, halo, methyl, phenoxy, or butoxy;

the benzohydroxamic acid is substituted at the meta and para positions by hydroxy, methoxy, amino, dimethylamino, halo, methyl, phenoxy, and/or butoxy.

In another generalization, each of the foregoing embodiments and subembodiments excludes benzohydroxamic acid, halobenzohydroxamic acid (especially chloro- and even more especially 3-chloro), and/or salicylhydroxamic acid.

Properties of the Compounds of the Present Invention

In the present invention, one or more of three in vitro bioassays can be utilized to evaluate the efficacy and toxicity of candidate skin-lightening compounds. The three bioassays characterize the compounds with regard to mammalian tyrosinase enzyme inhibition (cell free), pigmentation in cultured melanocyte cells, and cytotoxicity of mammalian cultured cells. Both cell-based pigmentation and cell-free enzymatic assays have been developed [5, 6, 25] using the mammalian melanocyte cell line, Mel-Ab, a C57BL/6 mouse-derived cell line that produces high levels of melanin. [21] A distinct advantage of this approach is that humans share substantial sequence similarities in their genes (DNA) and proteins (such as tyrosinase) with mice, relative to non-mammalian species (e.g., mushrooms). So, in vitro mouse Mel-Ab melanocytes can serve as adequate surrogates for human melanocytes and Mel-Ab-derived tyrosinase may substitute for the human enzyme for many pharmacologic purposes.

These adherent murine melanocytes are grown on tissue culture plastic in medium supplemented with fetal bovine serum, 12-O-tetradecanoylphorbol-13-acetate (TPA) to stimulate cell division via down-regulation of protein kinase C, [22, 23] and cholera toxin to stimulate adenylate cyclase activity in the absence of α-MSH. [15, 24] Cellular lysates of Mel-Ab cells may be used as tyrosinase enzyme preparations. Multi-well plate assays have been validated [5, 6, 25] for enzyme inhibition (e.g., DOPA oxidation by calorimetric measurement or radiolabeled substrate incorporation into melanin) and for pigmentation assays on cultured Mel-Ab cells. After 4-6 days of treatment of cultured cells, melanin content is determined using a spectrophotometer at 400+ nm. [6, 25] This assay can detect an apparent loss in pigmentation resulting from either inhibition of de novo synthesis (e.g. via inhibition of tyrosinase, or the adenylate cyclase pathway, or another pathway) or a cytostatic/cytotoxic mechanism. It is therefore a broad primary screen. It is used in parallel with the tyrosinase enzymatic assay to determine whether an inhibitor of pigmentation at the cellular level is acting primarily at the enzyme level.

To determine cytotoxicity (and/or cytostasis), crystal violet or other staining methods may be used to quantify adherent cell numbers following a period of treatment by an agent. HQ is typically used as a positive control in the assay, since it exhibits an IC₅₀ in the low micrograms per milliliter range on Mel-Ab culture using this assay, albeit owing to cytotoxicity and not inhibition of pigmentation per se. [6] It should be noted that some inhibitors identified in cell-free enzymatic assays might have subsequent difficulties with toxicity or delivery in melanocyte cell-based assays. Therefore, all three in vitro assays in combination provide an excellent characterization of candidate skin lightening compounds.

A distinct advantage of the screening systems (developed by the inventors of the present invention) is the focus on mammalian tyrosinase, as opposed to non-mammalian enyzmes often used by other investigators, such as mushroom tyrosinase. Since the biochemical and pharmacologic characteristics of an enzyme or isozyme can vary dramatically between species of organisms (e.g., due to dissimilarities in primary, secondary, and tertiary structure), it is highly preferable that candidate topical skin lighteners intended for human use be discovered based on their biochemical action against a mammalian source of the enzyme. Mushroom tyrosinase (and in some instances plant polyphenol oxidases) has been used in the vast majority of prior inhibitor studies. [28, 29] Yet fungal tyrosinase exhibits substantial dissimilarities from mammalian tyrosinase(s), and is viewed as a considerably inferior strategy for pharmacologic screening. Thus, the methods reported by the inventors of the present invention for screening against mammalian tyrosinase or within melanocytes is highly preferred over other possible screening strategies. [5, 6, 25]

The substrate kinetic “affinity” of mammalian tyrosinase for L-tyrosine is approximately K_{M}=600 μM. A potentially effective candidate skin lightening agent is considered to be desirable, active, and/or functional if it renders 50% inhibition of mammalian tyrosinase enzyme activity, at concentrations below half the enzyme's “affinity” for tyrosine in cell-free enzyme extracts (IC₅₀≦300 μM) and pigment production in melanocyte cell cultures (IC₅₀≦300 μM). In preferred embodiments the agent has an IC₅₀ against tyrosinase in cell-free enzyme extracts of less then 200, 100, 50, or 25 μM, and/or an IC₅₀ against pigment production in melanocyte cell cultures of less than 200, 100, 50, or 25 μM. In addition, it is desirable for the compounds to exhibit minimal cytotoxicity and/or cytostasis, e.g., thus retaining viability of 50% or more of the cultured cells (IC₅₀≧300 μM), as evidenced by adherent cell number. In preferred embodiments the agent exhibits toxicity at greater than 500, 750, or 1000 μM.

Curto, E. V., et al. (1999) [25] reports that methyl gentisate is an “effective” candidate skin-lightening agent based on in vitro bioassays, because it has an IC₅₀ of 67 μM (11.2±4 ug/mL) against tyrosinase in cell-free assays, an IC₅₀ of 184 μM (30.9±5 ug/mL) in pigmentation inhibition in melanocyte cell cultures, and a melanocyte cytotoxicity IC₅₀ of 707 μM (118.7±12 ug/mL). Methyl gentisate thus serves as an in vitro screening standard, against which the efficacy and cytotoxicity of other tyrosinase-inhibiting compounds can be evaluated. By contrast to methyl gentisate, hydroquinone is an inferior standard, exhibiting potent melanocyte cytotoxicity and minimal enzymatic inhibition. [5, 6, 25]

Significantly, many of the particular compounds of this invention are comparable to or are more effective candidate skin lightening agents than methyl gentisate. Thus, in another embodiment the invention provides methods for inhibiting pigment production that includes administering an effective treatment amount of a pigment-inhibiting compound wherein (i) the compound inhibits tyrosinase activity equivalent to or greater than methyl gentisate in cell-free enzyme extracts from mammalian melanocyte or melanoma cells, when evaluated using either a colorometric DOPA oxidation or a radiolabeled tyrosine or DOPA substrate assay as described in Curto, E. V., et al. (1999) [25], or (ii) the compound inhibits de novo pigment production (synthesis and/or accumulation) equivalent to or greater than methyl gentisate when evaluated in cultured mammalian melanocyte or melanoma cells. Curto, E. V., et al. (1999) [25]. In a preferred embodiment the toxicity of the compound in mammalian melanocyte, melanoma, or other cell cultures is equivalent to or less than the toxicity of methyl gentisate. Curto, E. V., et al. (1999) [25].

In another embodiment computer-based programs or models can aid in the understanding and predictability of structure-activity relationships, such that other effective compounds can be synthesized, identified, and evaluated. Examples of computer-based methodologies may include COMFA analysis or molecular orbital calculations, e.g., see Sakurada, J., et al., (1990) [26]. Coupling the computer-based SAR or other predictions with repetition(s) of the organic synthesis/bioassay cycle can identify benzohydroxamic acid derivatives with desirable features.

Definitions and Use of Terms

The following definitions and term construction are intended, unless otherwise indicated:

Specific and preferred values listed below for radicals, substituents, and ranges, are for illustration only; they do not exclude other defined values or other values within defined ranges for the radicals and substituents.

Halo is fluoro, chloro, bromo, or iodo.

Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to.

The term alkyl, as used herein, unless otherwise specified, refers to a saturated straight, branched, or cyclic, primary, secondary, or tertiary hydrocarbon of C₁ to C₁₀, and specifically includes methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. When the context of this document allows alkyl to be substituted, the moieties with which the alkyl group can be substituted are selected from the group consisting of hydroxyl, amino, alkylamino, arylamino, alkoxy, aryloxy, aryl, heterocycle, halo, carboxy, acyl, acyloxy, amido, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term lower alkyl, as used herein, and unless otherwise specified, refers to a C₁ to C₄ saturated straight, branched, or if appropriate, a cyclic (for example, cyclopropyl) alkyl group, including both substituted and unsubstituted forms. Unless otherwise specifically stated in this application, when alkyl is a suitable moiety, lower alkyl is preferred. Similarly, when alkyl or lower alkyl is a suitable moiety, unsubstituted alkyl or lower alkyl is preferred.

The terms alkenyl and alkynyl refer to alkyl moieties, including both substituted and substituted forms, wherein at least one saturated C—C bond is replaced by a double or triple bond. Thus, (C₂-C₆)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl. Similarly, (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl.

The term “—(CH₂)ₙ—” represents a saturated alkylidene radical of straight chain configuration. The term “n” can be any whole integer, including 0, 1, 2, 3, 4, 5, 6, 7, 8, 9, or 10. The moiety “—(CH₂)ₙ—” thus represents a bond (i.e., when n=0), methylene, 1,2-ethanediyl or 1,3-propanediyl, etc.

The term aryl, as used herein, and unless otherwise specified, refers to phenyl, biphenyl, or naphthyl, and preferably phenyl. The aryl group can be optionally substituted with one or more moieties selected from the group consisting of hydroxyl, acyl, amino, halo, carboxy, carboxamido, carboalkoxy, alkylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis,” John Wiley and Sons, Second Edition, 1991.

The term heteroaryl or heteroaromafic, as used herein, refers to an aromatic or unsaturated cyclic moiety that includes at least one sulfur, oxygen, nitrogen, or phosphorus in the aromatic ring. Nonlimiting examples are furyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, pyridazinyl, pyrazinyl, cinnolinyl, phthalazinyl, quinoxalinyl, xanthinyl, hypoxanthinyl, and pteridinyl. Functional oxygen and nitrogen groups on the heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acycl groups such as acetyl and propionyl, methanesulfonyl, and p-toluenelsulfonyl. The heteroaryl or heteroaromatic group can be optionally substituted with one or more moieties selected from the group consisting of hydroxyl, acyl, amino, halo, alkylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis,” John Wiley and Sons, Second Edition, 1991.

The term heterocyclic refers to a saturated nonaromatic cyclic group which may be substituted, and wherein there is at least one heteroatom, such as oxygen, sulfur, nitrogen, or phosphorus in the ring. The heterocyclic group can be substituted in the same manner as described above for the heteroaryl group.

The term acyl refers to a carboxylic acid ester in which the non-carbonyl moiety of the ester group is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

The term alkoxy, as used herein, and unless otherwise specified, refers to a moiety of the structure —O-alkyl, wherein alkyl is as defined above.

The term “pharmaceutically acceptable cation” is used herein to mean hydrogen and the nontoxic cations based on the alkali and alkaline earth metals, such as sodium, lithium, potassium, calcium, magnesium and the like, as well as those based on nontoxic ammonium, quaternary ammonium, and amine cations, including but not limited to ammonium, tetramethylammonium, tetraethylammonium, methylamino, dimethylamino, trimethylamino, triethylamino, and ethyl amino cations, and the like.

Synthetic Methods

A number of methods of synthesizing hydroxamic acids have earlier been reported. These methods general relate to the conversion of methyl esters of carboxylic acids to hydroxamic acids via the formation of potassium hydroxamate salt, (Hauser, C. R.; Rendrow, W. B. Org. Synth. Coll. Vol. II 1943, 67; Wise, M. M.; Brandt, W. W. J. Am. Chem. Soc. 1955, 77, 1058) conversion of acid chlorides to hydroxamic acids using hydroxylamine hydrochloride in the presence of sodium bicarbonate (Shukla, J. P.; Agrawal, Y. K.; Kuchya, K. P. J. Ind. Chem. Soc. 1974, 437), photolysis of azides followed by treatment with water (Horna, L.; Bauer, G.; Dorges, J. Chem. Ber. 1965, 98, 2631), and intramolecular photorearrangement of alkane nitronate anions (Yamada, K.; Kanakiya, T.; Naruchi, K.; Yammamoto, M. J. Am. Chem. Soc. 1981, 103, 7003).

Scheme 1 below illustrates the preparation of benzohydroxamic acid and its derivatives by the reaction of acids, acid esters and acid chlorides with hydroxylamine or hydroxylamine derivatives [29-32]:

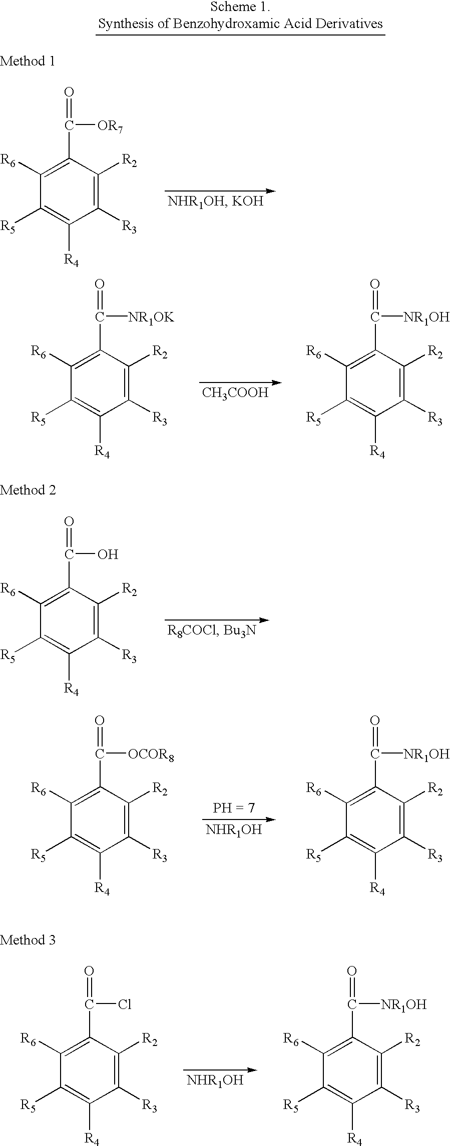

Pharmaceutical Formulations and Dosing Regimes

In one embodiment, a compound of this invention is applied or administered to the skin during an appropriate period and using a sufficient number of dosages to achieve skin lightening. The concentration of active compound in the composition will depend on absorption, inactivation, and excretion rates of the compound as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered as a single dose, or may be divided into a number of smaller doses to be administered at varying intervals of time.

Topical and other formulations of these active and/or functional compounds are of utility in lightening skin pigmentation in humans and other animals. These formulations may be useful for pure cosmetic purposes, simply to obtain a lighter skin color for perceived beautification. The formulations also have medicinal value and can, for example, decrease hyperpigmentation of melasma, age spots, freckles, and other skin blemishes. The compounds of this invention act primarily by inhibiting mammalian melanocyte tyrosinase, the rate-limiting enzyme in the production of melanin from tyrosine and DOPA. Some compounds also absorb ultraviolet radiation (UVR), and may thus protect skin from UVR and photoaging. In addition, some compounds may be antioxidants that protect skin from oxidative damage, and/or may prevent oxidative decomposition of product formulations.

If desirable these formulations could also be used to reduce pigmentation in hair, albeit during the biosynthesis of hair, by blocking pigment production within the melanocytes of hair follicles. The formulations would likely not affect the already emerged pigmented portions of hair, unlike a bleaching agent.

The formulations useful in the present invention contain biologically effective amounts of the functional and/or active compound(s). A biologically effective amount of the active compound is understood by those skilled in the art to mean that a sufficient amount of the compound in the composition is provided such that upon administration to the human or animal by, for example, topical route, sufficient active agent is provided on each application to give the desired result. However, the biologically effective amount of the active compound is at a level that it is not toxic to the human or animal during the term of treatment. By a suitable biologically compatible carrier, when the compound is topically applied, it is understood that the carrier may contain any type of suitable excipient in the form of cosmetic compositions, pharmaceutical adjuvants, sunscreen lotions, creams, and the like. In one embodiment the active compound is administered in a liposomal carrier.

The active compound is administered for a sufficient time period to alleviate the undesired symptoms and the clinical signs associated with the condition being treated, or to achieve the level of desired skin lightening. The individual dosage, dosage schedule, and duration of treatment may be determined by clinical evaluations by those of skill in the art.

Solutions or suspensions for topical application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates; and agents for the adjustment of tonicity such as sodium chloride or dextrose. pH can be adjusted with acids or bases, such as hydrochloric acid or sodium hydroxide.

Suitable vehicles, carriers, or formulations for topical application are known, and include lotions, suspensions, ointments, oil-in-water emulsions, water-in-oil emulsions, creams, gels, tinctures, sprays, powders, pastes, and slow-release transdermal or occlusive patches. Thickening agents, emollients, and stabilizers can be used to prepare topical compositions. Examples of thickening agents include petrolatum, beeswax, xanthan gum, or polyethylene glycol, humectants such as sorbitol, emollients such as mineral oil, lanolin and its derivatives, or squalene. A number of solutions and ointments are commercially available, especially for dermatologic applications.

A typical lotion formulation can be formulated to contain a USP standard or: polyoxyethylene, ethanol, critic acid, sodium citrate, 1,3-butylene glycol, 2-ethoxymethyl-5-hydroxy-γ-pyrone, an antiseptic, and pure water. A typical cream formulation can be formulated to contain a USP standard or: polyethylene glycol monostearate, glycerin monostearate, stearic acid, behenyl alcohol, liquid paraffin, glyceryl trioctanoate, paraoxybenzoate, 1,3-butylene glycol, 2-ethoxymethyl-5-hydroxy-γ-pyrone, an antiseptic, and pure water. A typical ointment formulation can be formulated to contain a USP standard or: polyoxyethylene sorbitan monostearate, polyoxyethylene sorbitol tetraoleate, glycerin monostearate, glycerin, bleached bee's wax, paraffin, stearic acid, behenyl alcohol, liquid paraffin, 1,3-butylene glycol, citric acid, 2-ethoxymethyl-5-hydroxy-γ-pyrone, an antiseptic, and pure water.

The compounds can be provided in the form of pharmaceutically-acceptable salts. As used herein, the term “pharmaceutically-acceptable salts or complexes” refers to salts or complexes that retain the desired biological activity of the parent compound and exhibit minimal, if any, undesired toxicological effects. Examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acids, naphthalenedisulfonic acids, and polygalacturonic acid; (b) base addition salts formed with polyvalent metal cations such as zinc, calcium, bismuth, barium, magnesium, aluminum, copper, cobalt, nickel, cadmium, and the like, or with an organic cation formed from N,N-dibenzylethylene-diamine or ethylenediamine; or (c) combinations of (a) and (b); e.g., a zinc tannate salt or the like.

The compounds can be modified in order to enhance their usefulness as pharmaceutical compositions. For example, it is well know in the art that various modifications of the active molecule, such as alteration of charge, can affect water and lipid solubility and thus alter the potential for percutaneous absorption. The vehicle, or carrier, can also be modified to enhance cutaneous absorption, enhance the reservoir effect, and minimize potential irritancy or neuropharmacological effects of the composition. See, in general, Arndt, et al. [27].

Thus, the invention provides various formulations as topical skin lighteners containing the active and/or functional compounds described above. The invention further provides formulations as topical anti-oxidants containing the active and/or functional compounds described above. In still another embodiment the invention provides formulations as topical sunscreens containing the active and/or functional compounds described above. Such formulations can be made in combination with other active and/or functional ingredients used in skincare products (e.g. organic or inorganic sunscreen, antioxidant, anti-inflammatory, anti-erythema, antibiotic, antimicrobial, humectant, or other ingredients). Other ingredients can be formulated with the compounds to augment their effect, including but not limited to Vitamin C, Vitamin E, magnesium ascorbyl phosphate, aloe vera extract, and retinoic acids. In addition, alpha-hydroxy acids can be included to speed up the skin lightening process by exfoliating surface colored skin.

In another embodiment one compound of the present invention may be combined with: (a) one or more other compounds of the present invention; and/or (b) one or more other known inhibitors of melanocyte tyrosinase (e.g., methyl gentisate); and/or (c) one or more known skin lighteners, in order to form an admixture of active ingredients within a topical formulation. It is possible that a combination of active or functional ingredients within a single formulation may be effective and desirable in some circumstances.

The compounds of the present invention can also be formulated for alternative routes of administration other than topical application, including but not limited to general systemic, oral, intradermal, transdermal, occlusive patches, intravenous, or parenteral administration, and pharmaceutical compositions known generally to those skilled in the art.

The compounds can also be formulated along with other active and/or functional ingredients used in skincare products, depending on the intended use of the formulation. For example, the compounds can be formulated with organic or inorganic sunscreens, an antioxidant, an anti-inflammatory, an anti-erythema, an antibiotic, an antimicrobial, a humectant, or other ingredients.

The active and/or functional compounds described above may also be of use in inhibiting tyrosinase-like enzymes from non-mammalian species, for instance for use in the food science industry for the inhibition of enzymatic browning. [28, 29] Inhibition of plant polyphenol oxidases by agents described here may coincidentally have activity against these non-mammalian enzymes. Suitable formulations for spraying or treatment of fruits are known generally to those skilled in the art. Treatment by these formulations containing the enzyme inhibitors of the present invention might improve shelf life of plant or fungal foods.

The compounds and compositions of the present invention can also be provided in the form of a kit, including instructions for applying the composition dermally or topically, including a frequency for such application.

### EXAMPLES

A first class of compounds based upon the template compound benzohydroxamic acid were tested for tyrosinase inhibition by methods described in Curto, E. V., et al. (1999) [25]. Results of the tests are given in Examples 1-5.

### Example 1

### 2-Substituted Benzohydroxamic Acids

TABLE 1

| ID# | R₂ | R₃ | R₄ | R₅ | R₆ | R₁ | R | X | E | P | T |
|---|---|---|---|---|---|---|---|---|---|---|---|
| 483 | OCH₃ | H | H | H | H | H | C | H | 32 | 807 | 900 |
| 318 | NH₂ | H | H | H | H | H | C | H | 19 | 431 | 600 |
| 484 | NHCOCH₃ | H | H | H | H | H | C | H | >231 | >1000 | >1000 |
| 282 | OH | H | H | H | H | H | C | H | 3.7 | 405 | 974 |

### Example 2

### 3-Substituted Benzohydroxamic Acids

TABLE 2

| ID# | R₂ | R₃ | R₄ | R₅ | R₆ | R₁ | R | X | E | P | T |
|---|---|---|---|---|---|---|---|---|---|---|---|
| 478 | H | OCH₃ | H | H | H | H | C | H | 0.91 | 148 | 580 |
| 500 | H | OC₆H₆ | H | H | H | H | C | H | 0.16 | 234 | >300 |
| 482 | H | Cl | H | H | H | H | C | H | <0.25 | 40 | 577 |
| 481 | H | CH₃ | H | H | H | H | C | H | <0.25 | 60 | 522 |
| 461 | H | NH₂ | H | H | H | H | C | H | 4.0 | 225 | >1000 |
| 486 | H | NHCOCH₃ | H | H | H | H | C | H | 5.3 | 369 | >1000 |
| 629 | H | OC₇H₁₃O₂ | H | H | H | H | C | H | 14 | | |
| 634 | H | OC₃H₂NS | H | H | H | H | C | H | 22 | | |
| 637 | H | OC₈H₉O₂ | H | H | H | H | C | H | 6 | | |

### Example 3

### 4-Substituted Benzohydroxamic Acids

TABLE 3

| ID# | R₂ | R₃ | R₄ | R₅ | R₆ | R₁ | R | X | E | P | T |
|---|---|---|---|---|---|---|---|---|---|---|---|
| 480 | H | H | OCH₃ | H | H | H | C | H | 1.26 | 57 | 170 |
| 479 | H | H | OCH₃ | H | H | H | C | K | 1.67 | 43 | 64 |
| 497 | H | H | CH₃ | H | H | H | C | H | 0.29 | 45 | 160 |
| 476 | H | H | NH₂ | H | H | H | C | H | 0.34 | 64 | 550 |
| 498 | H | H | N(CH₃)₂ | H | H | H | C | H | 2.2 | 44 | 136 |
| 477 | H | H | OBu | H | H | H | C | H | 12 | 326 | >554 |
| 601 | H | H | OC₆H₆N | H | H | H | C | H | 6 | | |

### Example 4

### Di-Substituted Benzohydroxamic Acids

TABLE 4

| ID# | R₂ | R₃ | R₄ | R₅ | R₆ | R₁ | R | X | E | P | T |
|---|---|---|---|---|---|---|---|---|---|---|---|
| 456 | H | OCH₃ | OCH₃ | H | H | H | C | H | 15 | 576 | 641 |
| 458 | H | OCH₃ | OCH₃ | H | H | H | C | K | 23 | 637 | 624 |
| 462 | OH | H | OCH₃ | H | H | H | C | H | 3.6 | >1000 | >1000 |
| 474 | OH | H | H | COCH₃ | H | H | C | K | 51 | >1000 | >1000 |
| 499 | H | NH₂ | CH₃ | H | H | H | C | H | 6.4 | 177 | 747 |

### Example 5

### Others

TABLE 5

| ID# | R₂ | R₃ | R₄ | R₅ | R₆ | R₁ | R | X | E | P | T |
|---|---|---|---|---|---|---|---|---|---|---|---|
| 357 | H | H | H | H | H | H | C | H | 0.82 | 64 | 64 |
| 485 | H | CH₃ | H | H | H | CH₃ | C | H | 68 | >1000 | >1000 |
| 245 | H | H | — | H | H | H | N | H | 3.2 | 183 | 566 |

Inhibition [μM] as measured in three assays. Here “E” “E” [μM] is the concentration of compound that produces 50% inhibition in the cell-free mammalian tyrosinase enzyme assay is the concentration of compound that produces 50% inhibition in the cell-free mammalian tyrosinase enzyme assay. “P” represents the concentration of compound that produces 50% inhibition in the mammalian Mel Ab melanocyte culture pigment assay system. “T” is the concentration of compound that results in 50% reduction in cell number in the mammalian melanocyte culture toxicity assay system.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. Hearing V J Jr., “Monophenol monooxygenase (tyrosinase): Purification, properties, and reactions catalyzed.” Methods Enzymol 142:154-165, 1987.

2. Spritz R A et al., “Genetic-disorders of pigmentation,” Adv Hum Genet 22: 1-45, 1994.

3. Frenk E, “Treatment of melasma with depigmenting agents.” Melasma: New Approaches to Treatment, pp. 9-15. Martin Dunitz Ltd., London, 1995.

4. Dooley T P, “Is there room for a moderate level of regularity oversight?” In: Drug Discovery Approaches for Developing Cosmeceuticals: Advanced Skin Care and Cosmetic Products (Ed. Hori W), Chap. 1.4. International Business Communications, Southborough, Mass., 1997.

5. Dooley T P, “Topical skin depigmentation agents: Current products and discovery of novel inhibitors of melanogenesis.” J. Dermatol. Treat. 8: 275-279, 1997.

6. Dooley T P, et al., “Development of an in vitro primary screen for skin depigmentation and antimelanoma agents.” Skin Pharmacol. 7: 188-200, 1994.

7. Morse J L (Ed.), “An Abridgment of The New Funk & Wagnalls Encyclopedia,” The Universal Standard Encyclopedia, Vol, 10, pp. 3662-3663. Unicorn, New York, 1955.

8. Budavari S (Ed.), “Gentisic acid,” Merck Index, 11^{th} Edn, Abstract No. 4290, p. 688. Merck & Co., Rahway, N.J., 1989.

9. J-Hua L, et al., “Direct analysis of salicylic acid, salicyl acyl glucuronide, salicyluric acid and gentisic acid in human plasma and urine by high-performance liquid chromatography.” J. Chromatogr. [B] 675: 61-70, 1996.

10. Glatt H R, et al., “Multiple activation pathways of benzene leading to products with varying genotoxic characteristics.” Environ Health Perspect 82: 81-89, 1989.

11. Glatt H R, “Endogenous mutagens derived from amino acids.” Mutat. Res. 238: 235-243, 1990.

12. La Du B N, “Alcaptonuria and ochronotic arthritis.” Mol. Biol. Med. 8: 31-38, 1991.

13 Hearing V J, “Mammalian monophenol monooxygenase (tyrosinase): purification, properties, and reactions catalyzed.” Methods Enzymol. 142: 154-65, 1987.

14. Spritz R A, et al., “Genetic disorders of pigmentation.” Adv. Hum. Genet. 22: 1-45, 1994.

15. Hadley M E et al, “Melanotropic peptides for therapeutic and cosmetic tanning of the skin.” N Y Acad. Sci. 680: 424-39, 1993.

16. Sakai C et al, “Modulation of murine melanocyte function in vitro by agouti signal protein.” EMBO J. 16: 3544-52, 1997.

17. Dooley T P, “Recent advances in cutaneous melanoma oncogenesis research.” Onco. Res. 6: 1-9, 1994.

18. Benmaman O, et al., “Treatment and camouflaging of pigmentary disorders.” Clin. Dermatol. 6: 50-61, 1998.

19. Zaumseil R-P, et al., “Topical azelaic acid in the treatment of melasma: pharmacological and clinical considerations.” In: Castanet J, Frenk E, Gaupe K et al (Eds) Melasma: new approaches to treatment. Martin Dunitz: London, pp 16-40, 1995.

20. Schallreuter K U, “Epidermal adrenergic signal transduction as part of the neuronal network in the human epidermis.” J. Invest. Dermatol. 2: 37-40, 1997.

21. Bennett D C, et al., “A line of non-tumorigenic mouse melanocytes, syngeneic with the B16 melanoma and requiring a tumour promoter for growth.” Int. J. Cancer 349: 414-18, 1987.

22. Dooley T P et al., “Polyoma middle T abrogates TPA requirement of murine melanocytes and induces malignant melanoma.” Oncogene 3: 531-6, 1988.

23. Brooks G et al., “Protein kinase C down-regulation, and not transient activation, correlates with melanocyte growth.” Cancer Res. 51: 3281-8, 1991.

24. O'Keefe E, et al., “Cholera toxin mimics melanocyte stimulating hormone in inducing differentiation in melanoma cells.” Proc. Natl. Acad. Sci. USA 71: 2500-4, 1974.

25. Curto, E. V., et al., “Inhibitors of Mammalian Melanocyte Tyrosinase: In Vitro Comparisons of Alkyl Esters of Gentisic Acid with Other Putative Inhibitors.” Biochem. Pharmacol. 57: 663-672, 1999.

26. Sakurada, J., et al., “Kinetic and molecular orbital studies on the rate of oxidation of monosubstituted phenols and anilines by horseradish peroxidase compound II.” Biochemistry 29: 4093-4098, 1990.

27. Arndt, et al., “The Pharmacology of Topical Therapy”, Dermatology in General Medicine, 1987; T. B. Fitzpatrick, A. Z. Eisen, K. Wolff, I. M. Freedberg and K. F. Austen, eds., 3d ed., McGraw Hill, Inc., New York, pp. 2532-2540.

28. Lee, C. Y. and Whitaker, J. R. (Eds.) “Enzymatic Browning and its Prevention”, Pub. American Chemical Society, Washington, D.C., 1995.

29. Lerch, K. “Tyrosinase: Molecular and active-site structure.” In Lee, C. Y. and Whitaker, J. R. (Eds.) Enzymatic Browning and its Prevention. Pub. American Chemical Society, Washington, D.C., pp.64-80, 1995.

30. Mishima, H., et al., “Fine structural demonstration of tyrosinase activity in the retinal pigment epithelium of normal and PTU-treated chick embryos.” Albrecht Von Graefes Arch. Klin. Exp. Ophthalmol. 211: 1-10, 1979.

31. Dryja, T. P., et al., “Demonstration of tyrosinase in the adult bovine uveal tract and retinal pigment epithelium.” Invest. Ophthalmol. Vis. Sci. 17: 511-514, 1978.

32. Higashi, Y., et al., “Inhibition of tyrosinase reduces cell viability in catecholaminergic neuronal cells.” J. Neurochem. 75: 1771-1774, 2000.

33. Enohara T, Ochi S, Uhara H, hamano K, Sekya H, Sekida E, “Heat- and Light-resistant Polyamide Compositions with Good Colorfastness to Light for Automobile Interior Materials”. 1995; JP 07173389, 7 pp.

34. Inukai T, Kimura M, Mekata H, “Manufacture of Thermoplastic Resin pellets”. 2001; JP 2001030238, 6 pp.

35. Nishihata S, Tsuneyoshi M, Tanimoto T, “Vibration-absorbing Rubber Compositions”. 1992; JP 04023841, 6 pp.

36. Ingebrigstsen J, Flurkey W H, “Affinity and Hydrophobic Chromatography of Mushroom Tyrosinase”. Photochemistry 1988; 27:1593-1599.

37. Yamamoto S, “Melanin Formation Inhibitors Containing Heterocycles”. 1993; JP 05124925 and JP 05124922.

38. Goto S, “High-contrast Heat-developable Photographic Material and Laser Imaging Method for Printing Platemaking”. 2000; JP 2000352788, 47 pp.

39. Venkatesan A M, Grosu G T, Davis J M, Hu B, Cole D C, Baker J L, Jacobson M P, O'dell M R. “N-Hydroxy-2-(Alkyl, Aryl or Heteroaryl Sulfanyl or Sulfonyl)-3-Substituted Alkyl, Aryl or Heteroaryl Amides as Matrix Metalloproteinase Inhibitors”. 2001; US 6172057, 58 pp.

40. Grusch M, Fritzer-Szekeres M, Fuhrmann G, Rosenberger G, Luxbacher C, Elford, H L, Smid K, Peters G J, Szekeres T, Krupitza G, “Activation of Caspases and Induction of Apoptosis by Novel Ribonucleotide Reductase Inhibitors Amidox and Didox”. Exp Hematol 2001; 29:623-632.

41. Odake S, Morikawa T, Tsuchiya M, Imamura L, Kobashi K, “Inhibition of Helicobacter Pylori Urease Activity by Hydroxamic Acid Derivatives”. Biol Pharm Bull 1994; 17:1329-1332.

42. Summers J B, “Preparation of Arenehydroxamic Acids and Vinylogs as Lipoxygenase Inhibitors”. 1989; EP 199151, 20 pp.

43. Rich P R, Wiegand N K, Blum H, Moore A L, Bonner W D, “Studies on the Mechanism of Inhibition of Redox Enzymes by Substituted Hydroxamic Acids”. Biochim Biophys Acta 1978; 525:325-337.

44. Rueegg C, Lerch K, “Cobalt Tyrosinase: Replacement of the Binuclear Copper of Neurospora Tyrosinase by Cobalt”. Biochemistry 1981; 20:1256-1262.

45. Hall et al., Anti-Cancer Drugs (1992), 3(3), 273-280.

What is claimed is:

1) A method of inhibiting or preventing pigment production in a mammal comprising administering to the mammal an effective amount of a compound defined by structure (I), or a pharmaceutically acceptable salt thereof: 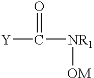
wherein:
M is a pharmaceutically acceptable cation;
R₁ is hydrogen, or C₁-C₆ alkyl or cycloalkyl; and
Y is substituted or unsubstituted cycloalkyl, aryl, heterocycle, or heteroaryl.

2) A method of inhibiting or preventing pigment production in a mammal comprising administering to the mammal an effective amount of a compound defined by structure (II), or a pharmaceutically acceptable salt thereof: 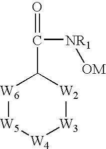
wherein:
M is a pharmaceutically acceptable cation;
R₁ is hydrogen, or C₁-C₆ alkyl or cycloalkyl;
W₂ is CR₂R_{2′}, NR², O or S; W₃ is CR₃R_{3′}, NR³, O or S; W₄ is CR₄R_{4′}, NR⁴, O or S; W₅ is CR₅R_{5′}, NR⁵, O or S; and W₆ is C₆NR_{6′}, O or S;
R₂, R₃, R₄, R₅, and R₆ are independently selected from (i) hydrogen, (ii) halogen, (iii) NO₂, (iv) —CN, (v) —OR₁₀ or phenoxy, (vi) —NHSO₂—C₁₋₃alkyl, (vii) —NHCO—C₁₋₅ alkyl, (viii) oxime, (ix) hydrazine, (x) —NR₉R₁₀, (xi) SO₂, (xii) SO₃, (xiii) SR₁₀, (xiv) C₁₋₅ acyloxy, (xv) PO₃, (xvi) PO₄, (xvii) thiol, (xviii) —COOR₉, (xix) C₂₋₅ alkynyl, (xx) C(O)C₁₋₃ alkyl, and (xxi) —C₁₋₈ alkyl, —C₂₋₈ alkenyl, aryl, heteroaryl, or heterocycle, optionally substituted with one or more of —OH, —SH, C(O)H, COOR₉, C₁₋₅ acyloxy, halogen, NR₉R₁₀, C₁₋₅ thioether, or C₁₋₅ alkoxy;
R_{2′}, R_{3′}, R_{4′}, R_{5′}, and R_{6′} are independently H or a valence for bonding;
R², R³, R⁴, R⁵, and R⁶ are independently selected from (i) substituted or unsubstituted alkyl, alkenyl, aryl, or heterocycle, (ii) —C₁₋₅ alkoxy, (iii) —OH, (iv) hydrogen, (v) C(O)—C₁₋₃ alkyl, (vi) —(CH₂)₁₋₅C(O)NR₉R₁₀, or (vii) a valence for bonding;
alternatively, R₃ and R₄, or R₄ and R₅, combine to form a fused ring-structure which is cycloalkyl, aryl, heterocyclyl or heteroaryl selected from phenyl, cyclopentyl, cyclohexyl, pyrrole, furan, thiophene, pyrazole, pyridine, —X—(CH₂)—X—, or —(CH₂)₂X— wherein X is independently NH, S, or O;
R₉ is hydrogen or C₁₋₃ alkyl;
R₁₀ is hydrogen, C₁₋₈ alkyl, —C₂₋₈ alkenyl, —(CH₂)ₙOₘ(CH₂)_{n′}-aryl, —(CH₂)ₙOₘ(CH₂)_{n′}-heteroaryl, or —(CH₂)ₙOₘ(CH₂)_{n′}-heterocycle, optionally substituted with one or more of —OH, —SH, C(O)H, COOR₉, C₁₋₈ acyloxy, halogen, NR₉R₉, C₁₋₅ thioether, or C₁₋₅ alkoxy;
m is 0 or 1; and
n and n′ are independently 0, 1, 2, or 3.

3) A method of inhibiting or preventing pigment production in a mammal comprising administering to the mammal an effective amount of a compound defined by structure (III), or a pharmaceutically acceptable salt thereof: 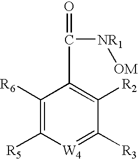
wherein:
M is a pharmaceutically acceptable cation;
R₁ is hydrogen, or C₁-C₆ alkyl or cycloalkyl;
W₄ is CR₄ or N;
R₂, R₃, R₄, R₅, and R₆ are independently selected from (i) hydrogen, (ii) halogen, (iii) NO₂, (iv) —CN, (v) —OR₁₀ or phenoxy, (vi) —NHSO₂-C₁₋₃alkyl, (vii) —NHCO—C₁₋₅ alkyl, (viii) oxime, (ix) hydrazine, (x) —NR₉R₁₀, (xi) SO₂, (xii) SO₃, (xiii) SR₁₀, (xiv) C₁₋₅ acyloxy, (xv) PO₃, (xvi) PO₄, (xvii) thiol, (xviii) —COOR₉, (xix) C₂₋₅ alkynyl, (xx) C(O)C₁₋₃ alkyl, and (xxi) —C₁₋₈ alkyl, —C₂₋₈ alkenyl, aryl, heteroaryl, or heterocycle, optionally substituted with one or more of —OH, —SH, C(O)H, COOR₉, C₁₋₅ acyloxy, halogen, NR₉R₁₀, C₁₋₅ thioether, or C₁₋₅ alkoxy;
alternatively, R₃ and R₄, or R₄ and R₅, combine to form a fused ring-structure which is cycloalkyl, aryl, heterocyclyl or heteroaryl selected from phenyl, cyclopentyl, cyclohexyl, pyrrole, furan, thiophene, pyrazole, pyridine, —X—(CH₂)—X—, or —(CH₂)₂X— wherein X is independently NH, S, or O;
R₉ is hydrogen or C₁₋₃ alkyl;
R₁₀ is hydrogen, C₁₋₈ alkyl, —C₂₋₈ alkenyl, —(CH₂)ₙOₘ(CH₂)_{n′}-aryl, —(CH₂)ₙOₘ(CH₂)_{n′}-heteroaryl, or —(CH₂)ₙOₘ(CH₂)_{n′}-heterocycle, optionally substituted with one or more of —OH, —SH, C(O)H, COOR₉, C₁₋₈ acyloxy, halogen, NR₉R₉, C₁₋₅ thioether, or C₁₋₅ alkoxy;
m is 0 or 1; and
n and n′ are independently 0, 1, 2, or 3.

4) A method of inhibiting or preventing pigment production in a mammal comprising administering to the mammal an effective amount of a compound defined by one of structures (IV)-(XXIV): 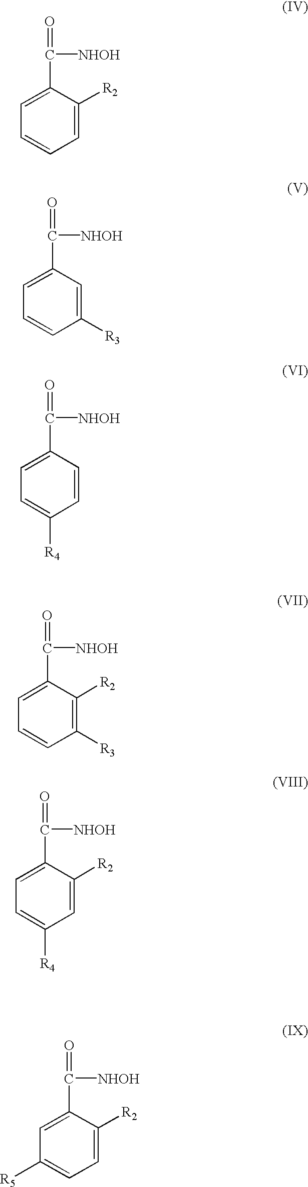 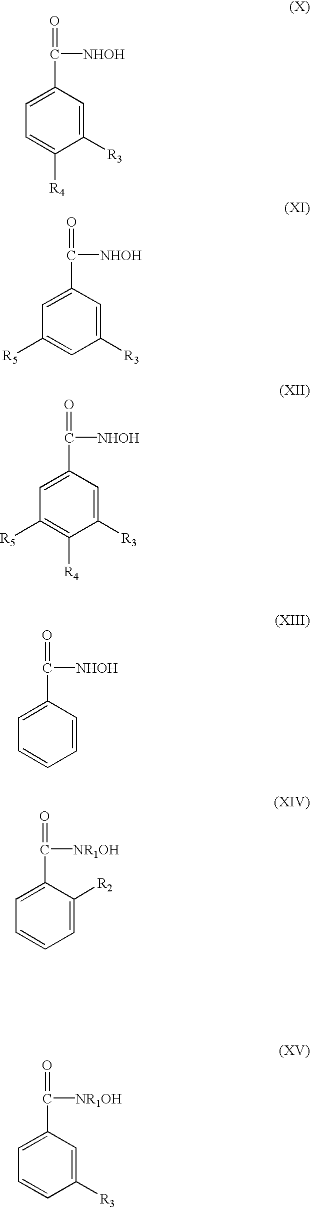 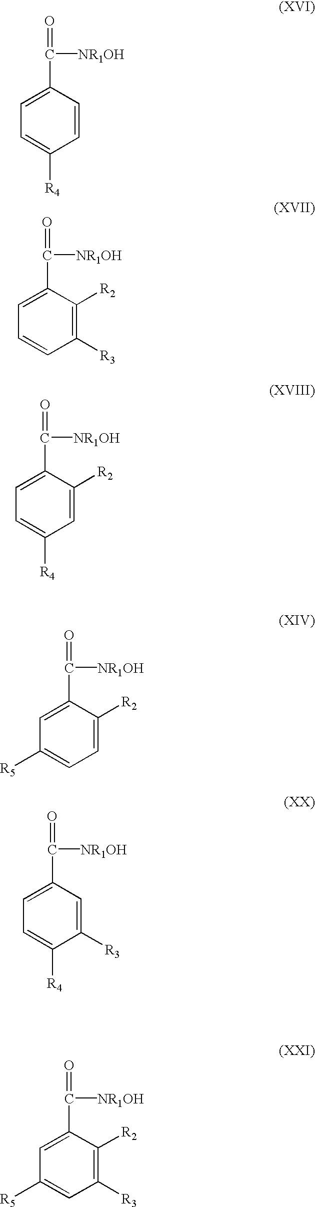 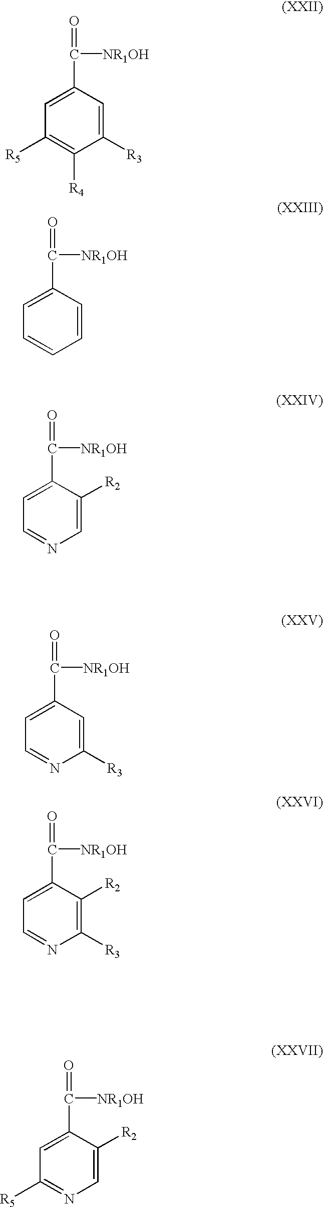 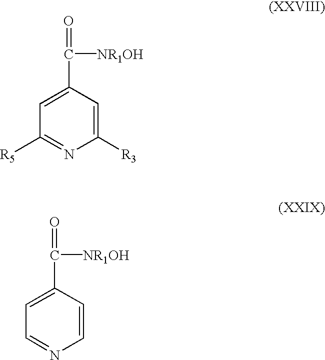
or a pharmaceutically acceptable salt, wherein:
R₁ is H or C₁-C₆ alkyl or cycloalkyl;
R₂, R₃, R₄, R₅, and R₆ are independently selected from (i) hydrogen, (ii) halogen, (iii) NO₂, (iv) —CN, (v) —OR₁₀ or phenoxy, (vi) —NHSO₂—C₁₋₃alkyl, (vii) —NHCO—C₁₋₅ alkyl, (viii) oxime, (ix) hydrazine, (x) —NR₉R₁₀, (xi) SO₂, (xii) SO₃, (xiii) SR₁₀, (xiv) C₁₋₅ acyloxy, (xv) PO₃, (xvi) PO₄, (xvii) thiol, (xviii) —COOR₉, (xix) C₂₋₅ alkynyl, (xx) C(O)C₁₋₃ alkyl, and (xxi) —C₁₋₈ alkyl, —C₂₋₈ alkenyl, aryl, heteroaryl, or heterocycle, optionally substituted with one or more of —OH, —SH, C(O)H, COOR₉, C₁₋₅ acyloxy, halogen, NR₉R₁₀, C₁₋₅ thioether, or C₁₋₅ alkoxy;
alternatively, R₃ and R₄, or R₄ and R₅, combine to form a fused ring-structure which is cycloalkyl, aryl, heterocyclyl or heteroaryl selected from phenyl, cyclopentyl, cyclohexyl, pyrrole, furan, thiophene, pyrazole, pyridine, —X—(CH₂)—X—, or —(CH₂)₂X— wherein X is independently NH, S, or O;
R₉ is hydrogen or C₁₋₃ alkyl;
R₁₀ is hydrogen, C₁₋₈ alkyl, —C₂₋₈ alkenyl, —(CH₂)ₙOₘ(CH₂)_{n′}-aryl, —(CH₂)ₙOₘ(CH₂)_{n′}-heteroaryl, or —(CH₂)ₙOₘ(CH₂)_{n′}-heterocycle, optionally substituted with one or more of —OH, —SH, C(O)H, COOR₉, C₁₋₈ acyloxy, halogen, NR₉R₉, C₁₋₅ thioether, or C₁₋₅ alkoxy;
m is 0 or 1; and
n and n′ are independently 0, 1, 2, or 3.

5) The method of claim 4 wherein
R₁ is hydrogen, or C₁-C₆ alkyl or cycloalkyl; and
R₂, R₃, R₄, R₅ and R₆ are independently selected from (i) hydrogen, (ii) halogen, (iii) NO₂, (iv) —CN, (v) —OR₁₀ or phenoxy, (vi) —NR₉R₁₀, (vii) C₁₋₅ acyloxy, (viii) thiol, (ix) COOR₉, (x) C(O)C₁₋₃alkyl, (xi) —NHCO—C₁₋₅ alkyl, and (xii) —C₁₋₅ alkyl, —C₂₋₅ alkenyl, aryl, -heteroaryl, or heterocycle, optionally substituted with one or more of —OH, —SH, C(O)H, COOR₉, C₁₋₅ acyloxy, halogen, NR₉R₁₀, C₁₋₅ thioether, or C₁₋₅ alkoxy.

6) The method of claim 4 wherein
R₁ is hydrogen or lower alkyl; and
R₂, R₃, R₄, R₅ and R₆ are independently selected from (i) hydrogen, (ii) halogen, (iii) NO₂, (iv) —CN, (v) —OR₉ or phenoxy, (v) —NR₉R₉, (vi) C₁₋₃ acyloxy, (vii) thiol, (viii) COOR₉, (x) C(O)C₁₋₃alkyl, (xi) —NHCO—C₁₋₃ alkyl, (xii) —C₁₋₃ alkyl, —C₂₋₃ alkenyl, aryl, heteroaryl, or heterocycle, optionally substituted with one or more of —OH, —SH, C(O)H, COOR₉, C₁₋₅ acyloxy, halogen, NR₉R₉, C₁₋₃ thioether, or C₁₋₃ alkoxy.

7) The method of claim 4 wherein
R₁ is hydrogen or lower alkyl; and
R₂, R₃, R₄, R₅ and R₆ are independently selected from (i) hydrogen, (ii) halogen, (iii) —OR₁₀ or phenoxy, (iv) —NR₉R₉, (v) thiol, (vi) C(O)C₁₋₃alkyl, (vii) —NHCO—C₁₋₃ alkyl, and (viii) —C₁₋₃ alkyl or C₂₋₃ alkenyl optionally substituted with one or more of —OH, —SH, halogen, and NH₂.

8) The method of claim 4 wherein
R₁ is hydrogen or lower alkyl; and
R₂, R₃, R₄, R₅ and R₆ are independently selected from lower alkyl, hydroxy, NR₉R₉, lower alkoxy, phenoxy, halo, NHC(O)CH₃, and acetyl.

9) The method of claim 4 wherein
R₁ is hydrogen or lower alkyl; and
R₂, R₃, R₄, R₅ and R₆ are independently selected from methyl, ethyl, methoxy, butoxy, phenoxy, hydroxy, NH₂, N(Me)₂, and halo.

10) The method of claim 4 wherein the compound is defined by structure (IV).

11) The method of claim 4 wherein the compound is defined by structure (V).

12) The method of claim 4 wherein the compound is defined by structure (VI).

13) The method of claim 4 wherein the compound is defined by structure (VII).

14) The method of claim 4 wherein the compound is defined by structure (VIII).

15) The method of claim 4 wherein the compound is defined by structure (IX).

16) The method of claim 4 wherein the compound is defined by structure (X).

17) The method of claim 4 wherein the compound is defined by structure (XI).

18) The method of claim 4 wherein the compound is defined by structure (XII).

19) The method of claim 4 wherein the compound is defined by structure (XIII).

20) The method of claim 4 wherein the compound is defined by structure (XIV).

21) The method of claim 4 wherein the compound is defined by structure (XV).

22) The method of claim 4 wherein the compound is defined by structure (XVI).

23) The method of claim 4 wherein the compound is defined by structure (XVII).

24) The method of claim 4 wherein the compound is defined by structure (XVIII).

25) The method of claim 4 wherein the compound is defined by structure (XIX).

26) The method of claim 4 wherein the compound is defined by structure (XX).

27) The method of claim 4 wherein the compound is defined by structure (XXI).

28) The method of claim 4 wherein the compound is defined by structure (XXII).

29) The method of claim 4 wherein the compound is defined by structure (XXIII).

30) The method of claim 4 wherein the compound is defined by structure (XXIV).

31) The method of claim 4 wherein the compound is defined by structure (XXV).

32) The method of claim 4 wherein the compound is defined by structure (XXVI).

33) The method of claim 4 wherein the compound is defined by structure (XXVII).

34) The method of claim 4 wherein the compound is defined by structure (XXVIII).

35) The method of claim 4 wherein the compound is defined by structure (XXIX).

36) The method of claim 4 wherein the compound is defined by structure (V), (VI), or (X), and R₃ and R₄ are methyl.

37) The method of claim 4 wherein the compound is defined by structure (V), (VI), or (X), and R₃ and R₄ are methoxy.

38) The method of claim 4 wherein the compound is defined by structure (V), (VI), or (X), and R₃ and R₄ are hydroxy.

39) The method of claim 4 wherein the compound is defined by structure (V), (VI), or (X), and R₃ and R₄ are NH₂.

40) The method of claim 4 wherein the compound is defined by structure (V), (VI), or (X), and R₃ and R₄ are N(Me)₂.

41) The method of claim 4 wherein the compound is defined by structure (V), (VI), or (X), and R₃ and R₄ are halo.

42) The method of claim 4 wherein the compound is defined by structure (V), (VI), or (X), and R₃ and R₄ are butoxy.

43) The method of claim 4 wherein the compound is defined by structure (V), (VI), or (X), and R₃ and R₄ are phenoxy.

44) The method of claim 4 wherein the compound is selected from the following, or a pharmaceutically acceptable salt thereof:
benzohydroxamic acid;
2-methoxybenzohydroxamic acid;
4-methoxybenzohydroxamic acid;
potassium salt of 4-methoxybenzohydroxamic acid;
4-methylbenzohydroxamic acid;
3-methoxybenzohydroxamic acid;
3-phenoxybenzohydroxamic acid;
3-chlorobenzohydroxamic acid;
3-methylbenzohydroxamic acid;
3,N-dimethylbenzohydroxamic acid;
3-aminobenzohydroxamic acid.
3-acetamidobenzohydroxamic acid.
3-aminobenzohydroxamic acid.
4-amino-4-methylbenzohydroxamic acid;
4-aminobenzohydroxamic acid.
4-dimethylaminobenzohydroxamic acid;
2-aminobenzohydroxamic acid;
2-acetamidobenzohydroxamic acid;
salicylhydroxamic acid;
4-butoxybenzohydroxamic acid;
3,4-dimethoxybenzohydroxamic acid;
potassium salt of 3,4-dimethoxybenzohydroxamic acid;
2-hydroxy-4-methoxybenzohydroxamic acid;
potassium salt of 2-hydroxy-5-acetylbenzohydroxamic acid; and
isonicotinohydroxamic acid.

45) The method of claim 4 wherein the compound is the following, or a pharmaceutically acceptable salt thereof:
3-methoxybenzohydroxamic acid.

46) The method of claim 4 wherein the compound is the following, or a pharmaceutically acceptable salt thereof:
3-phenoxybenzohydroxamic acid.

47) The method of claim 4 wherein the compound is the following, or a pharmaceutically acceptable salt thereof:
3-chlorobenzohydroxamic acid.

48) The method of claim 4 wherein the compound is the following, or a pharmaceutically acceptable salt thereof:
3-methylbenzohydroxamic acid.

49) The method of claim 4 wherein the compound is the following, or a pharmaceutically acceptable salt thereof:
3-aminobenzohydroxamic acid.

50) The method of claim 4 wherein the compound is the following, or a pharmaceutically acceptable salt thereof:
3-amino-4-methyl-benzohydroxamic acid.

51) The method of claim 4 wherein the compound is the following, or a pharmaceutically acceptable salt thereof:
4-aminobenzohydroxamic acid.

52) The method of claim 4 wherein the compound is the following, or a pharmaceutically acceptable salt thereof:
isonicotinohydroxamic acid.

53) The method of claim 1 wherein the mammal is a human.

54) The method of claim 2 wherein the mammal is a human.

55) The method of claim 3 wherein the mammal is a human.

56) The method of claim 4 wherein the mammal is a human.

57) A topical skin treatment pharmaceutical composition comprising a compound defined by one of structures (IV)-(XXIX): 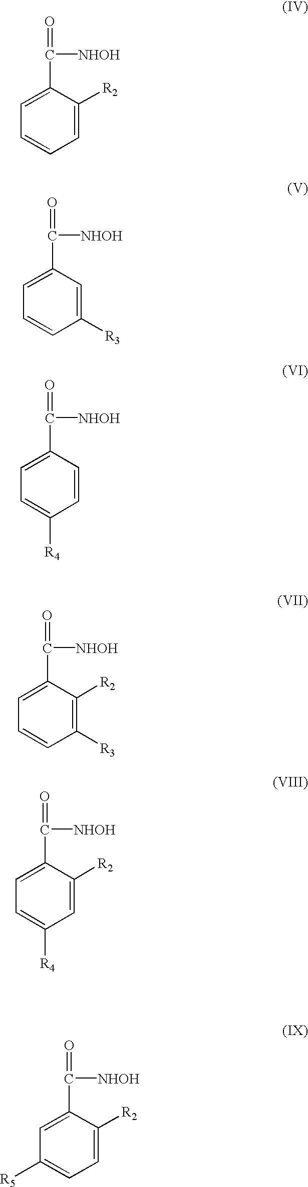 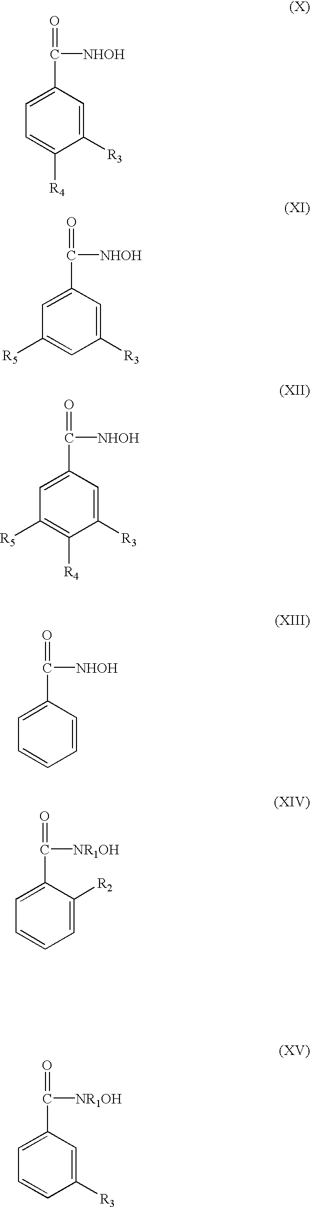 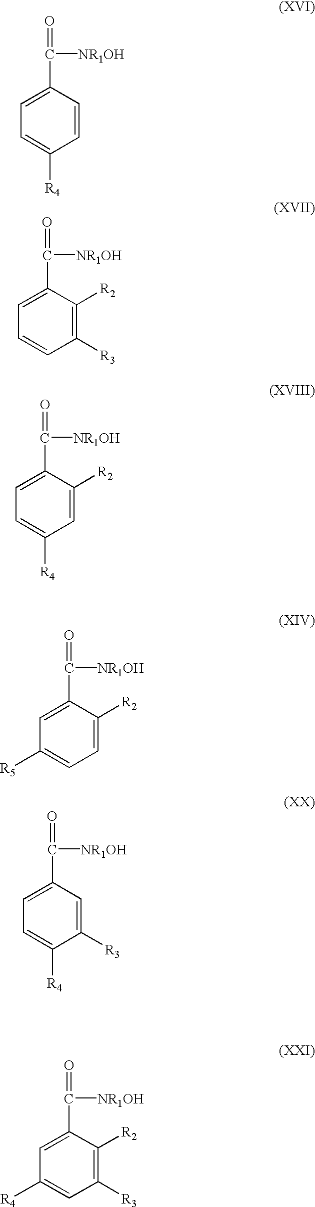 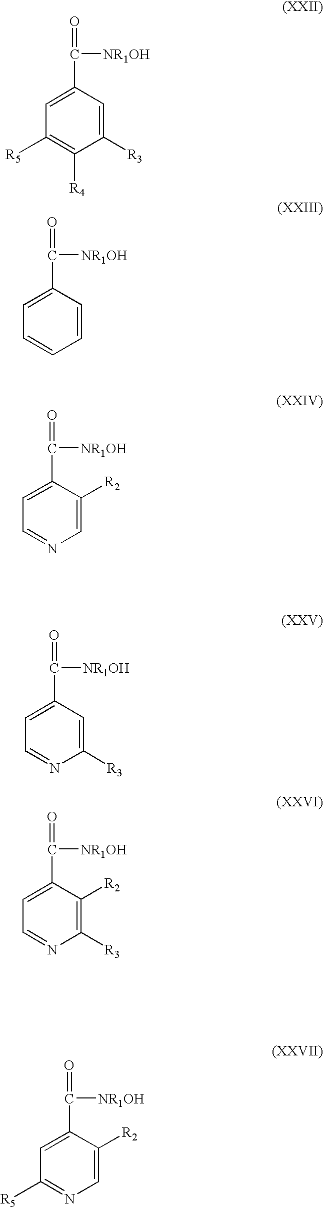 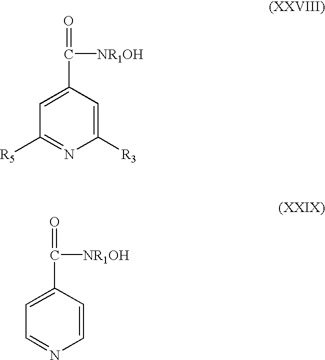
or a pharmaceutically acceptable salt thereof, wherein:
R₁ is hydrogen, or C₁-C₆ alkyl or cycloalkyl;
R₂, R₃, R₄, R₅, and R₆ are independently selected from (i) hydrogen, (ii) halogen, (iii) NO₂, (iv) —CN, (v) —OR₁₀ or phenoxy, (vi) —NHSO₂-C₁₋₃alkyl, (vii) —NHCO—C₁₋₅ alkyl, (viii) oxime, (ix) hydrazine, (x) —NR₉R₁₀, (xi) SO₂, (xii) SO₃, (xiii) SR₁₀, (xiv) C₁₋₅ acyloxy, (xv) PO₃, (xvi) PO₄, (xvii) thiol, (xviii) —COOR₉, (xix) C₂₋₅ alkynyl, (xx) C(O)C₁₋₃ alkyl, and (xxi) —C₁₋₅ alkyl, —C₂₋₈ alkenyl, aryl, heteroaryl, or heterocycle, optionally substituted with one or more of —OH, —SH, C(O)H, COOR₉, C₁₋₅ acyloxy, halogen, NR₉R₁₀, C₁₋₅ thioether, or C₁₋₅alkoxy;
alternatively, R₃ and R₄, or R₄ and R₅, combine to form a fused ring-structure which is cycloalkyl, aryl, heterocyclyl or heteroaryl selected from phenyl, cyclopentyl, cyclohexyl, pyrrole, furan, thiophene, pyrazole, pyridine, —X—(CH₂)—X—, or —(CH₂)₂X— wherein X is independently NH, S, or O;
R₉ is hydrogen or C₁₋₃ alkyl;
R₁₀ is hydrogen, C₁₋₈ alkyl, —C₂₋₈ alkenyl, —(CH₂)ₙOₘ(CH₂)_{n′}-aryl, —(CH₂)ₙOₘ(CH₂)_{n′}-heteroaryl, or —(CH₂)ₙOₘ(CH₂)_{n′}-heterocycle, optionally substituted with one or more of —OH, —SH, C(O)H, COOR₉, C₁₋₈ acyloxy, halogen, NR₉R₉, C₁₋₅ thioether, or C₁₋₅alkoxy;
m is 0 or 1; and
n and n′ are independently 0, 1, 2, or 3.

58) The topical skin treatment pharmaceutical composition of claim 57 comprising a compound selected from the following, or a pharmaceutically acceptable salt thereof:
benzohydroxamic acid;
2-methoxybenzohydroxamic acid;
4-methoxybenzohydroxamic acid;
potassium salt of 4-methoxybenzohydroxamic acid;
4-methylbenzohydroxamic acid;
3-methoxybenzohydroxamic acid;
3-phenoxybenzohydroxamic acid;
3-chlorobenzohydroxamic acid;
3-methylbenzohydroxamic acid;
3,N-dimethylbenzohydroxamic acid;
3-aminobenzohydroxamic acid.
3-acetamidobenzohydroxamic acid.
3-aminobenzohydroxamic acid.
4-amino-4-methylbenzohydroxamic acid;
4-aminobenzohydroxamic acid.
4-dimethylaminobenzohydroxamic acid;
2-aminobenzohydroxamic acid;
2-acetamidobenzohydroxamic acid;
salicylhydroxamic acid;
4-butoxybenzohydroxamic acid;
3,4-dimethoxybenzohydroxamic acid;
potassium salt of 3,4-dimethoxybenzohydroxamic acid;
2-hydroxy-4-methoxybenzohydroxamic acid;
potassium salt of 2-hydroxy-5-acetylbenzohydroxamic acid; and
isonicotinohydroxamic acid.

59) A compound selected from the following, or a pharmaceutically acceptable salt thereof:
2-methoxybenzohydroxamic acid;
3-methoxybenzohydroxamic acid;
3-phenoxybenzohydroxamic acid;
3-methylbenzohydroxamic acid;
3,N-dimethylbenzohydroxamic acid;
3-acetamidobenzohydroxamic acid;
3-amino-4-methylbenzohydroxamic acid;
2-aminobenzohydroxamic acid;
2-acetamidobenzohydroxamic acid;
2-hydroxy-4-methoxybenzohydroxamic acid; and
potassium salt of 2-hydroxy-5-acetylbenzohydroxamic acid.
